SEQUENCE LISTING (40 sequences)

SEQ ID NO: 1 - DNA, 903 bp, Homo sapiens
atggagggggtgttgtacaagtggaccaactatctcacaggctggcagcctcgttggtttgttttagataatggaatcttatcctactatgattcacaagatgatgtttgcaaagggagcaaaggaagcataaagatggcagtttgtgaaattaaagttcattcagcagacaacacaagaatggaattaatcattcctggagagcagcatttctacatgaaggcagtgaatgcagctgaaagacagaggtggctggtcgctctggggagctccaaagcatgtttgactgatacaaggactaaaaaagaaaaagaaataagtgaaaccagtgaatcgctgaaaaccaaaatgtctgaacttcgcctctactgtgacctcttaatgcagcaagttcatacaatacaggaatttgttcaccatgatgagaatcattcatctcctagtgcagagaacatgaatgaagcctcttctctgcttagtgccacgtgtaatacattcatcacaacgcttgaggaatgtgtgaagatagccaatgccaagtttaaacctgagatgtttcaactgcaccatccggatcccttagtttctcctgtgtcaccttctcctgttcaaatgatgaagcgttctgtcagccaccctggttcttgcagttcagagaggagtagccactctataaaagaaccagtatctacacttcaccgactctcccagcgacgccgaagaacctactcagatacagattcttgtagtgatattcctcttgaagacccagatagacctgttcactgttcaaaaaatacacttaatggagatttggcatcagcaaccattcctgaagaaagcagacttacggccaaaaaacaatctgaatcagaagatactcttccatccttctcttcctga

SEQ ID NO: 2 - protein, 300 aa, Homo sapiens
MEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDTRTKKEKEISETSESLKTKMSELRLYCDLLMQQVHTIQEFVHHDENHSSPSAENMNEASSLLSATCNTFITTLEECVKIANAKFKPEMFQLHHPDPLVSPVSPSPVQMMKRSVSHPGSCSSERSSHSIKEPVSTLHRLSQRRRRTYSDTDSCSDIPLEDPDRPVHCSKNTLNGDLASATIPEESRLTAKKQSESEDTLPSFSS

SEQ ID NO: 3 - DNA, 349 bp, Artificial Sequence
  Synthetic nucleotide sequence encoding synthetic FAPP1 including attB sites used for Gateway homologous recombination cloning
acaagtttgtacaaaaaagcaggctccatggaaggtgttctgtataaatggacaaattacctgaccggctggcagccacgttggtttgttctggataacggtattctgtcttattacgattcacaagatgatgtgtgtaaaggtagcaaaggcagtatcaagatggctgtttgcgaaatcaaggtgcattctgcagataatacccgtatggaactgattattcctggcgaacagcatttctatatgaaagctgttaacgctgcagaacgtcaacgctggctggttgctctgggttcttcaaaagcgtgtctgaccgatactcgcacccagctttcttgtacaaagtggt

SEQ ID NO: 4 - protein, 99 aa, Artificial Sequence
  Synthetic FAPP1 including attB sites used for Gateway homologous recombination cloning
MEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDTR

SEQ ID NO: 5 - DNA, 1914 bp, Artificial Sequence
  Snythetic nucleic acid sequence encoding GST-FAPP1-GFP fusion protein, as present in the pSDK1 vector
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccggaattcccgggtcgactcgagacaagtttgtacaaaaaagcaggctccatggaaggtgttctgtataaatggacaaattacctgaccggctggcagccacgttggtttgttctggataacggtattctgtcttattacgattcacaagatgatgtgtgtaaaggtagcaaaggcagtatcaagatggctgtttgcgaaatcaaggtgcattctgcagataatacccgtatggaactgattattcctggcgaacagcatttctatatgaaagctgttaacgctgcagaacgtcaacgctggctggttgctctgggttcttcaaaagcgtgtctgaccgatactcgcacccagctttcttgtacaaagtggtccctcgaggacgatgacgataaggatatgtccggctatccatatgacgtcccagactatgctggctccatgggatccggaattcaaaggcctacgtcgacgagctcactagtcgcggcggcggccaccatgagcaagggcgaggaactgttcactggcgtggtcccaattctcgtggaactggatggcgatgtgaatgggcacaaattttctgtcagcggagagggtgaaggtgatgccacatacggaaagctcaccctgaaattcatctgcaccactggaaagctccctgtgccatggccaacactggtcactaccttctcttatggcgtgcagtgcttttccagatacccagaccatatgaagcagcatgactttttcaagagcgccatgcccgagggctatgtgcaggagagaaccatctttttcaaagatgacgggaactacaagacccgcgctgaagtcaagttcgaaggtgacaccctggtgaatagaatcgagctgaagggcattgactttaaggaggatggaaacattctcggccacaagctggaatacaactataactcccacaatgtgtacatcatggccgacaagcaaaagaatggcatcaaggtcaacttcaagatcagacacaacattgaggatggatccgtgcagctggccgaccattatcaacagaacactccaatcggcgacggccctgtgctcctcccagacaaccattacctgtccacccagtctgccctgtctaaagatcccaacgaaaagagagaccacatggtcctgctggagtttgtgaccgctgctgggatcacacatggcatggacgagctgtacaagcatcatcatcatcatcattga

SEQ ID NO: 6 - protein, 637 aa, Artificial Sequence
  Synthetic GST-FAPP1-GFP fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFPGRLETSLYKKAGSMEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDTRTQLSCTKWSLEDDDDKDMSGYPYDVPDYAGSMGSGIQRPTSTSSLVAAAATMSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYKHHHHHH

SEQ ID NO: 7 - DNA, 1908 bp, Artificial Sequence
  Synthetic nucleic acid encoding GST-FAPP1-mCherry fusion protein, as present in pSDK2 vector
atgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggttccgcgtggatccccggaattcccgggtcgactcgagacaagtttgtacaaaaaagcaggctccatggaaggtgttctgtataaatggacaaattacctgaccggctggcagccacgttggtttgttctggataacggtattctgtcttattacgattcacaagatgatgtgtgtaaaggtagcaaaggcagtatcaagatggctgtttgcgaaatcaaggtgcattctgcagataatacccgtatggaactgattattcctggcgaacagcatttctatatgaaagctgttaacgctgcagaacgtcaacgctggctggttgctctgggttcttcaaaagcgtgtctgaccgatactcgcacccagctttcttgtacaaagtggtccctcgaggacgatgacgataaggatatgtccggctatccatatgacgtcccagactatgctggctccatgggatccggaattcaaaggcctacgtcgacgagctcactagtcgcggcggcggccaccatggtttctaaaggtgaagaagataacatggctatcatcaaagaatttatgcgtttcaaggttcatatggaaggttctgttaacggtcatgaatttgaaattgaaggtgaaggtgaaggtcgtccatatgaaggtactcagaccgctaaactgaaagttaccaaaggtggtccactgccatttgcttgggatattctgtctccacagtttatgtatggttctaaggcttacgttaaacatccagctgatattccagattatctgaaactgtcttttccagaaggttttaaatgggaacgtgttatgaattttgaagatggtggtgttgttaccgttacccaggattcttctctgcaggatggtgaatttatttataaagttaaactgcgtggtactaattttccatctgatggtccagttatgcagaaaaagactatgggttgggaagcatcttctgaacgtatgtatccagaagatggtgctctgaaaggtgaaattaaacagcgtctgaaactgaaagatggtggtcattatgatgctgaagttaaaaccacctataaagctaaaaaaccagttcagctgccaggtgcttacaacgttaacatcaaactggatatcacctctcataacgaagattacaccatcgttgaacagtatgaacgtgctgaaggtcgtcattctaccggtggtatggatgaactgtataaacatcatcatcatcatcattga

SEQ ID NO: 8 - protein, 635 aa, Artificial Sequence
  Synthetic GST-FAPP1-mCherry fusion protein
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPEFPGRLETSLYKKAGSMEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDTRTQLSCTKWSLEDDDDKDMSGYPYDVPDYAGSMGSGIQRPTSTSSLVAAAATMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKHHHHHH

SEQ ID NO: 9 - DNA, 2175 bp, Arabidopsis thaliana
atgtctaaggtagtgtacgaaggatggatggttaggtatggaaggaggaagatcggacgatcgtatattcatatgaggtattttgtgttggagcctcgtcttttggcgtattacaagaagaaacctcaggattatcaggttcctatcaagaccatgttaattgatggtaactgcagagttgaggatcgaggcttgaaaacacatcatggacatatggtttacgttttgtctgtctataacaaaaaagaaaagagtcatagaattacgatggcagcgttcaacattcaggaagcactaatgtggaaggaaaaaattgagtctgttatagaccagcatcaagagtcccaagttccaaatggtcagcagtatgtttcatttgaatataaatctggaatggatactggaaggactgcttcatcttcagaccatgaaagccaatctgcaatttcctttagatttagtgcggcagaggatgaagaggactctcgacgtagcttaatgaggaggacaactattggaaatggtcctccagaatctgtacttgactggaccaaagaatttgatgcagagttggcgaaccagaattccgacaaccaagcattttccaggaaacactggcgtctccttcagtgccaaaatggtcttcggatttttgaagagcttcttgaagttgattaccttccaagaagctgtagcagggcaatgaaggctgttggtgtagtggaggcaacatgtgaggaaatattcgagcttctgatgagcatggacggcactcgttatgagtgggactgcagctttcagtttggtagcttagtggaagaggtcgatggtcacacagcagtcctctatcacagacttctgctcgactggtttccaatgattgtgtggcctcgcgacctctgttatgtccgctattggcgccgtaatgatgatgggagttatgttgtgttgttccgttctagggagcatgagaattgtggtccacaacctggatgtgttcgggctcatcttgagagtggaggatataatatttccccactaaagcctcggaatgggaggcctagaacgcaagtgcaacatctaatacaaattgatctaaaagggtggggtgcaggctatcttccagcatttcaacaacattgtcttcttcaaatgctgaatagtgttgctggtttgcgggaatggttttcacagacagatgagagaggtgttcatacccggatccctgtcatggttaatatggcatcatcttccttgagcttgactaagagtgggaagtctctgcataagtctgccttttctcttgatcaaacaaattccgttaacagaaactccttgcttatggatgaagactcggatgatgatgatgaatttcagattgctgaatcagaacaagagcctgaaacaagtaaaccagagaccgatgtcaagagaccagaagaagaacctgctcacaacattgatctgtcatgcttctcgggtaacctaaagcgcaatgaaaacgaaaacgcccgtaactgctggaggatttctgatggtaacaacttcaaagttcggggcaagaatttcggtcaagagaaaagaaagatacctgctggcaagcatcttatggatctcgttgctgttgactggttcaaagacagtaaaagaatagatcatgttgctagacgcaaaggctgcgcagcacaagttgctgcagaaaaaggtctattctcaatggtggtaaatgttcaagttccaggatcaacacactacagtatggtgttttatttcgtgatgaaagaactagtacctgggtccctcttgcaacggtttgtggatggtgatgatgaattccgaaatagtaggctaaagcttataccattagttcctaagggctcatggatagtacggcaaagtgtgggaagcaccccttgtctccttgggaaagcagtggactgcaactacatccgtggcccgacatacttagaaattgacgtagatattggttcatcaaccgttgcaaatggagtgctcggcctcgtcattggcgtaatcacgtctctggttgtggaaatggctttccttgtacaggcaaatacagcggaagaacagccagagaggcttatcggtgcagttcgggtttcgcatattgagctctcttcggctatagttccgaatctggagtcagaataa

SEQ ID NO: 10 - protein, 724 aa, Arabidopsis thaliana
MSKVVYEGWMVRYGRRKIGRSYIHMRYFVLEPRLLAYYKKKPQDYQVPIKTMLIDGNCRVEDRGLKTHHGHMVYVLSVYNKKEKSHRITMAAFNIQEALMWKEKIESVIDQHQESQVPNGQQYVSFEYKSGMDTGRTASSSDHESQSAISFRFSAAEDEEDSRRSLMRRTTIGNGPPESVLDWTKEFDAELANQNSDNQAFSRKHWRLLQCQNGLRIFEELLEVDYLPRSCSRAMKAVGVVEATCEEIFELLMSMDGTRYEWDCSFQFGSLVEEVDGHTAVLYHRLLLDWFPMIVWPRDLCYVRYWRRNDDGSYVVLFRSREHENCGPQPGCVRAHLESGGYNISPLKPRNGRPRTQVQHLIQIDLKGWGAGYLPAFQQHCLLQMLNSVAGLREWFSQTDERGVHTRIPVMVNMASSSLSLTKSGKSLHKSAFSLDQTNSVNRNSLLMDEDSDDDDEFQIAESEQEPETSKPETDVKRPEEEPAHNIDLSCFSGNLKRNENENARNCWRISDGNNFKVRGKNFGQEKRKIPAGKHLMDLVAVDWFKDSKRIDHVARRKGCAAQVAAEKGLFSMVVNVQVPGSTHYSMVFYFVMKELVPGSLLQRFVDGDDEFRNSRLKLIPLVPKGSWIVRQSVGSTPCLLGKAVDCNYIRGPTYLEIDVDIGSSTVANGVLGLVIGVITSLVVEMAFLVQANTAEEQPERLIGAVRVSHIELSSAIVPNLESE

SEQ ID NO: 11 - protein, 80 aa, Raphanus sativus
MAKFASIIVLLFVALVVFAAFEEPTMVEAQKLCQRPSGTWSGVCGNNNACKNQCIRLEKARHGSCNYVFPAHKCICYFPC

SEQ ID NO: 12 - protein, 50 aa, Dahlia merckii
ELCEKASKTWSGNCGNTGHCDNQCKSWEGAAHGACHVRNGKHMCFCYFNC

SEQ ID NO: 13 - DNA, 624 bp, Candida albicans
atgaataacgaatacgactatttatttaaattattattgattggtgattcaggtgtcggtaaatcctgtttgttattgagatttgctgacgacacatatacaccagattatatttctaccattggtgttgattttaaaatcagaaccatcgagttggacggcaaaactatcaaattacaaatctgggacactgctggtcaagaaaggtttagaactatcacttcttcctattatagaggggcacatggtatcattatcgtgtacgatgtgactgaccaggaatcatttaataatgtcaaacaatggttacaagaaatcgaccgttatgccacaggtggtgtcatgaaattattagttggtaataaggctgatttgtctgataaaaaaatcgtcgaatatactgctgctaaagaatttgctgatgccttggacattccatttttagaaacctccgctttatcatcgaccaatgttgaacaagctttttacactatggcaagacaaatcaaagcccaaatgacaaacaatgccaatgccggaaatgctgccaatgccaagggcaaatctaatgtgaatttgagaggtgaatctttgacttctaaccaatcgaattcctgttgttaa

SEQ ID NO: 14 - protein, 207 aa, Candida albicans
MNNEYDYLFKLLLIGDSGVGKSCLLLRFADDTYTPDYISTIGVDFKIRTIELDGKTIKLQIWDTAGQERFRTITSSYYRGAHGIIIVYDVTDQESFNNVKQWLQEIDRYATGGVMKLLVGNKADLSDKKIVEYTAAKEFADALDIPFLETSALSSTNVEQAFYTMARQIKAQMTNNANAGNAANAKGKSNVNLRGESLTSNQSNSCC

SEQ ID NO: 15 - DNA, 624 bp, Candida albicans
atgaataacgaatacgactatttatttaaattattattgattggtgattcaggtgtcggtaaatcctgtttgttattgagatttgctgacgacacatatacaccagattatatttctaccattggtgttgattttaaaatcagaaccatcgagttggacggcaaaactatcaaattacaaatctgggacactgctggtcaagaaaggtttagaactatcacttcttcctattatagaggggcacatggtatcattatcgtgtacgatgtgactgaccaggaatcatttaataatgtcaaacaatggttacaagaaatcgaccgttatgccacaggtggtgtcatgaaattattagttggtattaaggctgatttgtctgataaaaaaatcgtcgaatatactgctgctaaagaatttgctgatgccttggacattccatttttagaaacctccgctttatcatcgaccaatgttgaacaagctttttacactatggcaagacaaatcaaagcccaaatgacaaacaatgccaatgccggaaatgctgccaatgccaagggcaaatctaatgtgaatttgagaggtgaatctttgacttctaaccaatcgaattcctgttgttaa

SEQ ID NO: 16 - protein, 207 aa, Candida albicans
MNNEYDYLFKLLLIGDSGVGKSCLLLRFADDTYTPDYISTIGVDFKIRTIELDGKTIKLQIWDTAGQERFRTITSSYYRGAHGIIIVYDVTDQESFNNVKQWLQEIDRYATGGVMKLLVGIKADLSDKKIVEYTAAKEFADALDIPFLETSALSSTNVEQAFYTMARQIKAQMTNNANAGNAANAKGKSNVNLRGESLTSNQSNSCC

SEQ ID NO: 17 - DNA, 2106 bp, Phytophthora sojae
atgagctcgcgcggagcttcacaggacgccgcctctgccgggcgcgcgtccgaggagaaggcgggcgacggcgtgtacgtgaccaagaaccgctcgctcttctccatgtggctgcacggcaaggcggtgcccaaccgcgcgcacccggctgtggtcttccgctcggcggacgtgatccaggagggctacctgctcaagcagggcctgcgcctcaagatgtggtcgcgccgctacttcatcctgcggctcgaggagcgccacatgacgctcggctactacacgagcaaggactcgctcacgctgtgctccgagacgcccatcggccccggccacgtgctggaccacgtcaacacggccaagtacccgcgccgcctggagctgcgctgcggcaccaaggtcatgacgctcgaggccgaggaccagaaggcctacgaggcctggaagagcgcgctgcaggaggccatccgctggaaccacgccatggtgcccaccaaggacggcagcttcgtcacgtacggcaagcaggcgtccgaggacctcaagcaggaggagcgcagccgcgccgaggccgccaagaagctgcgcgagaagcagcgcgcggacgaggcggcggcggccgccaacgcggcgaacaagcccaagtacctgccggccacgcgccccggcacgcagtgcttcatgacgtccaacacgcggttcgagatcccgtccaacttcgagtacgtcaagaccattggctcgggcgcgtacggagtcgtgatctctgctacagactccaagagcggcaagacggtggccatcaagaacatccagcgcgcgttcgacgacctgacggacgccaagcgcatcgtgcgcgagattaagttgatgaggcacctgaaccacaagtgcgtgctgggggtggaggacatcttcgagcccgtggcgctggacaagttcgaggacgtgtacatcgtgtcgcagctcatggccacggacctgcatcgagtaatctactcgagacacgcgctgtcggacgagcacatcgccttcttcatgtaccagatgctgtgtgccatgaagtacgtgcactcggccaatgtgatccaccgcgacctgaagccgtcgaatgtgctggtgaacgccaactgtgagctcaagatctgcgactttggactggcgaggggagttttccccgaggaggagctggagctgacggagtatgtggtcacgcggtggtaccgcgcgcccgagattatgctgggatgcatgaagtacactcgcgaagtggacgtctggtccatgggctgcatctttgccgagatgatgtcgcgcaagccgctcttccccggacaggactacattgaccagctgcacctaatcatgaacgcgctcggagctccgaacgaccaggagctgtacttcttgaccaacgcgcgcgcacggaagttcatgaacgccgagttccagaagcgcggaccgaacccgaccaagcccctcgcgcacatgttcacggactcgccgccggacgcgctggatctgctgcagaagatgctggtgatcgacccgaacaagcgaatcagcgtggacgaggcgctcgcacacccgtacctggcctctatccgcaatatggacgacgagaccatggccacgtccagctttgacttcgactttgagaacgagaagctcacgaagcccgtgctgcagagactcatctgggaggaaatgcggcatttccacccgatcgagggcgaggagccgggtgtcagctctaacgccgctgctgagggagagaatgacagctttgcgactacacaggcatccaacacccccgtgacgcccgtgactccagccactgctgagcaggataatacatcttcctcctcttcttcttcggaggctacgggtactgctacgaccgctgaggtggaagtggaggtgacgacgcctgccacggaagaagcaaggccggaagacgacggcgaagccagcaccagaccaactggcgacgacaaacagagtaccaatagtgaccagaaaatcgttaggacaagcgtcggcagcgacaacccgacagacgctcagacgcggcaagaggctggcgagccggcacgtgaagtcgcataa

SEQ ID NO: 18 - protein, 701 aa, Phytophthora sojae
MSSRGASQDAASAGRASEEKAGDGVYVTKNRSLFSMWLHGKAVPNRAHPAVVFRSADVIQEGYLLKQGLRLKMWSRRYFILRLEERHMTLGYYTSKDSLTLCSETPIGPGHVLDHVNTAKYPRRLELRCGTKVMTLEAEDQKAYEAWKSALQEAIRWNHAMVPTKDGSFVTYGKQASEDLKQEERSRAEAAKKLREKQRADEAAAAANAANKPKYLPATRPGTQCFMTSNTRFEIPSNFEYVKTIGSGAYGVVISATDSKSGKTVAIKNIQRAFDDLTDAKRIVREIKLMRHLNHKCVLGVEDIFEPVALDKFEDVYIVSQLMATDLHRVIYSRHALSDEHIAFFMYQMLCAMKYVHSANVIHRDLKPSNVLVNANCELKICDFGLARGVFPEEELELTEYVVTRWYRAPEIMLGCMKYTREVDVWSMGCIFAEMMSRKPLFPGQDYIDQLHLIMNALGAPNDQELYFLTNARARKFMNAEFQKRGPNPTKPLAHMFTDSPPDALDLLQKMLVIDPNKRISVDEALAHPYLASIRNMDDETMATSSFDFDFENEKLTKPVLQRLIWEEMRHFHPIEGEEPGVSSNAAAEGENDSFATTQASNTPVTPVTPATAEQDNTSSSSSSSEATGTATTAEVEVEVTTPATEEARPEDDGEASTRPTGDDKQSTNSDQKIVRTSVGSDNPTDAQTRQEAGEPAREVA

SEQ ID NO: 19 - DNA, 1182 bp, Artificial Sequence
  Synthetic DNA sequence encoding SP-FAPP1-GFP fusion protein
atggggtacatgtgcattaagatttcgttttgtgtgatgtgtgtgttggggttggtgatcgtgggtgatgttgcctacgctcaggatacaagtttgtacaaaaaagcaggctccatggaaggtgttctgtataaatggacaaattacctgaccggctggcagccacgttggtttgttctggataacggtattctgtcttattacgattcacaagatgatgtgtgtaaaggtagcaaaggcagtatcaagatggctgtttgcgaaatcaaggtgcattctgcagataatacccgtatggaactgattattcctggcgaacagcatttctatatgaaagctgttaacgctgcagaacgtcaacgctggctggttgctctgggttcttcaaaagcgtgtctgaccgatactcgcacccagctttcttgtacaaagtggtcggatccccgggtaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 20 - protein, 393 aa, Artificial Sequence
  Amino acid sequence of synthetic SP-FAPP1-GFP fusion protein
MGYMCIKISFCVMCVLGLVIVGDVAYAQDTSLYKKAGSMEGVLYKWTNYLTGWQPRWFVLDNGILSYYDSQDDVCKGSKGSIKMAVCEIKVHSADNTRMELIIPGEQHFYMKAVNAAERQRWLVALGSSKACLTDTRTQLSCTKWSDPRVPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - DNA, 1812 bp, Saprolegnia parasitica
atgatggagccggcctcggaaccgccggcgaccgccgaccggacctcgacgacgtcggcgccgggcgacgatggcatgtacgtcacgcggaagcggtcggccttctccaagtggctgcatggcaagggtatctcgtcgtcggccaagcacccggcgcccgtcttccggtccgccgacgtcatccaggaaggctacatgctcaagcaaggctcgcgcttccgcatgtggacgaagcggtactttatcttgcgcttggaagagaagcacatgacgctcggctactacacgtccaaggaagagctcatcttgtgctccgagacgccgatcggacccggccatgcgcttctcgacttgagcaaggacggccggttccgcttggagctgcggagcggaacgcgctcgatggtggtcgagatcgagctcgaggccgactatgtcaagtggaagaacgcgctccaggaagccgtgcgatggcaccagaccatggtctttgacggcgccaacaaggtcacgtcgtatggcaaaaagtgcattgacgacgaagcagcggaagccgaagcccgtcttgcggccgccaagaagcagcgcgagaccgcggccaagaaggtcgaggacgcggcgacggccaacgccaacaagcccaagttcctcccctcgacgcgcaagggccatcagtgctttacggtctccaacacgaagtttgagatcccactacagtacacgtactccaagacgattgggtcgggcgcctacggcgtcgtgatcgccgccgagttggaccgacccgccgacgtggatggcgcctcgaaccttgtcgcgatcaagaacattcagcgcgcgttcgacgacctcacggacgccaagcgcatcgtgcgcgagatcaagctcatgcgccatctgcagcacaagtccctcctggggatcacggacattctcgagcccgtgagcttggacgcgttcgaagacgtctacattgtctcggagtgcatggccacggacctccaccgcgtcatctactcgcgccacgcgctctcggaagagcacatttgcttcttcttgtaccagatgctcgcggcgctcaagtacattcactcggccaacgtaattcatcgcgatctcaagccgagcaacattttggtgaacgccaactgcgagctcaagatttgcgactttggccttgcccgcggcgtccaagaggacttggagctcacggaatatgtcgtgacgcgctggtaccgcgcgcccgagatcatgctcggctgcatcaagtacacgcaccatgtcgacgtgtggagcctcggctgtatctttgccgaaatgctgagccgcaagccgctcttccccggccaagactacattgaccaactgcatttgatcatgaacgcgcttggtgtgccgaccgacgacgagctctactttgtcaccaacatgcgcgcccgcaagtttatgaacaccgagtaccacacgcggggccgggaaccgctcaagccattgaatgccctctttgccgacattgcgccggacgccctcgacttgctcgagaagatgctcgtgctggacccaaacaagcgcatccgcgtcgacgatgcactcgcgcacccgtactttgcgtccattcgccaagtcgaggacgagacggtcgcgccgtccgtgtttgactttgagtttgagaaggaagacctcaacaagcggaggctgcaggagctcatttggggcgaaatgcgccactttcatccggcgccgcaaggtgtcgacgaacctttcctccaaaaaacaaccaacaacacggacgacgtgaaggcggccatgtag

SEQ ID NO: 22 - protein, 603 aa, Saprolegnia parasitica
MMEPASEPPATADRTSTTSAPGDDGMYVTRKRSAFSKWLHGKGISSSAKHPAPVFRSADVIQEGYMLKQGSRFRMWTKRYFILRLEEKHMTLGYYTSKEELILCSETPIGPGHALLDLSKDGRFRLELRSGTRSMVVEIELEADYVKWKNALQEAVRWHQTMVFDGANKVTSYGKKCIDDEAAEAEARLAAAKKQRETAAKKVEDAATANANKPKFLPSTRKGHQCFTVSNTKFEIPLQYTYSKTIGSGAYGVVIAAELDRPADVDGASNLVAIKNIQRAFDDLTDAKRIVREIKLMRHLQHKSLLGITDILEPVSLDAFEDVYIVSECMATDLHRVIYSRHALSEEHICFFLYQMLAALKYIHSANVIHRDLKPSNILVNANCELKICDFGLARGVQEDLELTEYVVTRWYRAPEIMLGCIKYTHHVDVWSLGCIFAEMLSRKPLFPGQDYIDQLHLIMNALGVPTDDELYFVTNMRARKFMNTEYHTRGREPLKPLNALFADIAPDALDLLEKMLVLDPNKRIRVDDALAHPYFASIRQVEDETVAPSVFDFEFEKEDLNKRRLQELIWGEMRHFHPAPQGVDEPFLQKTTNNTDDVKAAM

SEQ ID NO: 23 - DNA, 330 bp, Arabidopsis thaliana
atgtctaaggtagtgtacgaaggatggatggttaggtatggaaggaggaagatcggacgatcgtatattcatatgaggtattttgtgttggagcctcgtcttttggcgtattacaagaagaaacctcaggattatcaggttcctatcaagaccatgttaattgatggtaactgcagagttgaggatcgaggcttgaaaacacatcatggacatatggtttacgttttgtctgtctataacaaaaaagaaaagagtcatagaattacgatggcagcgttcaacattcaggaagcactaatgtggaaggaaaaaattgagtctgttatagac

SEQ ID NO: 24 - protein, 110 aa, Arabidopsis thaliana
MSKVVYEGWMVRYGRRKIGRSYIHMRYFVLEPRLLAYYKKKPQDYQVPIKTMLIDGNCRVEDRGLKTHHGHMVYVLSVYNKKEKSHRITMAAFNIQEALMWKEKIESVID

SEQ ID NO: 25 - DNA, 333 bp, Glycine max
atgtcgtcgaaggtggtgtacgaagggtggatggtgcggtacggccggcggaagatcggacgatcgttcatccacatgaggtacttcgtgttggagtcgcggctgctcgcgtattacaagaggaaaccgcaggataatcaggtaccaattaaaacactgctaattgatggcaactgtagagtggaggatagaggcttgaaggctcatcatggacatatggtttatgttttgttgttttacaacaagaaagataagaaccatcgaattatgatggcagcatttaacatccaagatatattgctctgcctcttggtatatttagtgtctatttat

SEQ ID NO: 26 - protein, 111 aa, Glycine max
MSSKVVYEGWMVRYGRRKIGRSFIHMRYFVLESRLLAYYKRKPQDNQVPIKTLLIDGNCRVEDRGLKAHHGHMVYVLLFYNKKDKNHRIMMAAFNIQDILLCLLVYLVSIY

SEQ ID NO: 27 - DNA, 396 bp, Solanum tuberosum
atgtcaaaagtggtgtatgatgggtggatggttaggtatgggcggaggaagattggcagatcctatattcacatgcggtattttgttctcgagacgcgattgttggcttactacaagagaaagcctcaggataatgtggttccaatcaagacacttcccatagatggtaattgtcgggtggaggaccgaggcctgaagactcatcatggacatatggtttatgtcttatcagtttacaacaagaaagacaagtataaccgtgtcacgatggcagctttcaacattcaggaggcacttttctggaaagaaaacattgaatcaatcattgatcagcatcaggcagtctcaagggaactcaatggccattcagtacaattcgtcttgaggtatacaatc

SEQ ID NO: 28 - protein, 132 aa, Solanum tuberosum
MSKVVYDGWMVRYGRRKIGRSYIHMRYFVLETRLLAYYKRKPQDNVVPIKTLPIDGNCRVEDRGLKTHHGHMVYVLSVYNKKDKYNRVTMAAFNIQEALFWKENIESIIDQHQAVSRELNGHSVQFVLRYTI

SEQ ID NO: 29 - DNA, 348 bp, Arabidopsis thaliana
ccacaggttcggcagcatatcattgatggtttcagccccaacgccttggacatgttcactagagagtttgacttctttgacaaggttacttctatatctggggtgttattccctcttccaaaggaagaacgcagagccggtattaggagggagttggagaaaattgaaatgcagggagatgacctttatttgccaactgctcctaacaagcttgttaggggtatccgtgtagacagtggaatacccttacaatcagctgccaaagtccctatcatgataacttttaacgttattgatcgtgatggtgaccacagtgatgtaaaaccacaggcttgcattttcaaggtt

SEQ ID NO: 30 - protein, 116 aa, Arabidopsis thaliana
PQVRQHIIDGFSPNALDMFTREFDFFDKVTSISGVLFPLPKEERRAGIRRELEKIEMQGDDLYLPTAPNKLVRGIRVDSGIPLQSAAKVPIMITFNVIDRDGDHSDVKPQACIFKV

SEQ ID NO: 31 - DNA, 348 bp, Glycine max
ccagctgtaaggcagcgtataattgacggttttaatccaaaggcacttgacatatttaaaagagagtttgatttctttgacaaagttacatccatctctggtgtactatttccacttcctaaagaagaacgccgagccggtatccgaagggagttggagaaaattgaaatggacggtgaggacctttatttaccgacagctcctaacaagcttgttaggggtattcgagtagatagtggtattcccttacaatcagccgcaaaagttccaatcatgataacttttaatgtaattgatcgagatggggacgaaaatgatgtaaagccacaagcttgcattttcaaggtt

SEQ ID NO: 32 - protein, 116 aa, Glycine max
PAVRQRIIDGFNPKALDIFKREFDFFDKVTSISGVLFPLPKEERRAGIRRELEKIEMDGEDLYLPTAPNKLVRGIRVDSGIPLQSAAKVPIMITFNVIDRDGDENDVKPQACIFKV

SEQ ID NO: 33 - DNA, 348 bp, Nicotiana tabacum
cctctcgtgaggcaacgaataattgatgggttcaatgaaaaggctcgtgatgtgtttcaaagggagttcgatttctttgacaaggttacatctatatctggcgcactctatccacttcccaaggaggagagaagagctggcattcggagggagctggagaaaattgagatgcaaggagatgatctctatctacctacagctcctaataaaatcgtgaaaggaattcacgttgatagtggaattcccttgcaatcagctgccaaggttcctattatgatcacatttaacgttgcagatcgagatggcgatcaaaatgacataaaaccccaagcttgtatattcaaggtt

SEQ ID NO: 34 - protein, 116 aa, Nicotiana tabacum
PLVRQRIIDGFNEKARDVFQREFDFFDKVTSISGALYPLPKEERRAGIRRELEKIEMQGDDLYLPTAPNKIVKGIHVDSGIPLQSAAKVPIMITFNVADRDGDQNDIKPQACIFKV

SEQ ID NO: 35 - DNA, 111 bp, Bombyx mori
aggtggaagatcttcaagaaaattgaaaaaatgggcaggaacattcgtgacggcatcgtcaaagcgggcccggcgatcgaggtccttggttcggctaaagctataggaaaa

SEQ ID NO: 36 - protein, 37 aa, Bombyx mori
RWKIFKKIEKMGRNIRDGIVKAGPAIEVLGSAKAIGK

SEQ ID NO: 37 - DNA, 438 bp, Arabidopsis thaliana
atggagagtatctggcgaatcgcgacgggacaagatccgagccgtgaagattacgaagggatcgagttctggtcaaaccctgagcgttctggttggctcacaaagcaaggcgattacatcaaaacctggcgtcgtcgttggttcgttctcaaacgagggaagcttctctggttcaaagatcaagccgctgctggaattcgtggatctacgccgcgtggtgtgatctccgttggtgattgtctcaccgtgaaaggagctgaggatgttgtgaataagccttttgcttttgagctatctagtggtagctataccatgttcttcattgctgataatgagaaggagaaagaagagtggattaattcgattggaagatcgattgtgcaacactcgaggtctgtgacggattctgaggttctcgattatgatcacaggcggtga

SEQ ID NO: 38 - protein, 145 aa, Arabidopsis thaliana
MESIWRIATGQDPSREDYEGIEFWSNPERSGWLTKQGDYIKTWRRRWFVLKRGKLLWFKDQAAAGIRGSTPRGVISVGDCLTVKGAEDVVNKPFAFELSSGSYTMFFIADNEKEKEEWINSIGRSIVQHSRSVTDSEVLDYDHRR

SEQ ID NO: 39 - DNA, 438 bp, Glycine max
atggcgagcctgtggcgcgcggcaaccggcatgacggacaacgccaccgattacgacggcgtcgagttctggtcgaaccccgagcggaccggctggctcaccaagcagggcgagtacatcaagacctggcgccgccgctggttcgtcctcaagcaaggcaagctcttctggttcaaggactccgccgtcacgcgtgcgtctcgtccacgcggcgtggtccccgtcgccacgtgcctcaccgtcaaaggcgccgaagacatcctcaacaagcccaacgccttcgagctctccacgcgctccgacaccatgtacttcatcgccgactccgagaaggagaaggaggattggatcaactctatcggccgctccatcgtccagcactccagatccgtcaccgattccgagatcatcgattacgataacaactcctccaagcgc

SEQ ID NO: 40 - protein, 146 aa, Glycine max
MASLWRAATGMTDNATDYDGVEFWSNPERTGWLTKQGEYIKTWRRRWFVLKQGKLFWFKDSAVTRASRPRGVVPVATCLTVKGAEDILNKPNAFELSTRSDTMYFIADSEKEKEDWINSIGRSIVQHSRSVTDSEIIDYDNNSSKR

(19) United States
(12) Patent Application Publication
(10) Pub. No.: US 2011/0165649 A1
(43) Pub. Date: Jul. 7, 2011

(54) METHODS AND COMPOSITIONS TO IMPROVE THE HEALTH OF PLANTS, ANIMALS AND MICROBES BY MANIPULATING PROTEIN ENTRY INTO SYMBIONTS AND THEIR HOSTS

(76) Inventors: Brett Tyler, Blacksburg, VA (US); Shiv Kate, Fairfax, VA (US)

(21) Appl. No.: 12/985,617

(22) Filed: Jan. 6, 2011

Related U.S. Application Data
(60) Provisional application No. 61/292,632, filed on Jan. 6, 2010.

(51) Int. Cl. C12N 5/10; C12N 9/96; C12N 5/02; C12N 1/00; C07K 14/00; C07K 14/415

(52) U.S. Cl. 435/188; 435/325; 435/375; 435/419; 435/243; 530/350; 530/370

(57) ABSTRACT

Fusion constructs with i) a domain that is specific for binding, on the surface of a cell, a lipid that is characteristic of the cell, and ii) a domain or agent that possesses an activity of interest that impacts the cell, are provided. Binding of the fusion construct to the characteristic lipid results in attachment of the construct to the cell and 1) expression of the activity of interest at the cell surface or, 2) entry of the construct into the cell, so that the activity of interest is expressed inside the cell. The cell may be a pathogen, cancer cell or other pathological cell displaying a characteristic lipid, and the domain or agent may be toxic or inhibitory to the cell. Alternatively, the cell may be non-pathogenic and the activity of interest may elicit a desired response from the cell, e.g. cell division, up regulation of a gene sequence, etc.

### CROSS-REFERENCE TO RELATED APPLICATIONS

This application claims benefit of U.S. provisional patent application 61/292,632, filed Jan. 6, 2010, the complete contents of which is hereby incorporated by reference.

### STATEMENT OF FEDERALLY SPONSORED RESEARCH AND DEVELOPMENT

This invention was made, in part, with government support under Grant No. IOS-0924861 awarded by The United States National Science Foundation. The government has certain rights in the invention.

### SEQUENCE LISTING

This application includes as the Sequence Listing the complete contents of the accompanying text file “Sequence.txt”, created Jan. 4, 2011, containing 74,830 bytes, hereby incorporated by reference.

### BACKGROUND OF THE INVENTION

1. Field of the Invention

The invention generally relates to compositions and methods for effecting the selective delivery of substances to a cell which has a unique or characteristic lipid on its outer surface. In particular, the invention provides fusion or composite constructs comprising i) a first domain that is specific or selective for binding a characteristic lipid on the surface of the cell; and ii) a domain or agent that possesses an activity of interest that has an effect on the cell. The presence of the characteristic lipid thus serves to recruit the construct to the cell, where the activity of interest is then expressed.

2. Background of the Invention

The delivery of substances to cells has been of interest for some time. In particular, specific or selective targeted delivery is of interest. Generally, efforts in this area have involved the identification of proteins on the surface of the cell which, when bound by a ligand, mediate the transfer of the ligand into the interior of the cell. However, despite intensive research efforts, there is still a need to identify additional means of targeted delivery of substances of interest into cells. This is particularly true with respect to pathogenic organisms, where it is highly desirable to deliver substances that are toxic or inhibitory to the pathogen in a manner that does not damage host cells.

Pathogens for which methods of prevention and treatment are needed include, for example, oomycetes, fungi, protozoa, nematodes and trematodes. Oomycetes are economically important organisms because many of them are aggressive plant and animal pathogens which cause hundreds of billions of dollars of losses each year. For example, the Phytophthora group of oomycetes causes diseases such as dieback, late blight in potatoes (the cause of the Great Hunger or Potato Famine of the 1840s in Ireland and other parts of Europe), the current problem of sudden oak death, rhododendron root rot, and ink disease in the American chestnut. Damping off caused by the Pythium group is a very common problem in greenhouses, where the organism kills newly emerged seedlings. Oomycete downy mildews and white blister rusts (e.g. Albuginales) cause diseases on a variety of flowering plants as well as on grapes (bringing about the near-devastation of vineyards in France in the 1870s), lettuce, corn, cabbage, and many other crop plants.

One Pythium species, Pythium insidiosum, is also known to infect mammals and is the causative agent of pythiosis. Pythiosis occurs most commonly in dogs and horses, but is also found in cats, cattle, and humans. Pythium typically occupies stagnant standing water such as swamps in late summer and infects animals who drink the water or who have open lesions that are exposed to the oomycete. Pythium insidiosum is different from other members of the genus in that human and horse hair, skin, and decaying animal and plant tissue are chemoattractants for its zoospores.

Some species of oomycetes grow on the scales or eggs of fish, and on amphibians. The water mold Saprolegnia causes lesions on fish and is especially problematic when water is stagnant, as in aquaria or on fish farms, or when fish are at high population densities, such as when salmon swim upstream to spawn. This oomycete is thus of major ecological and commercial importance.

Fungi are also major pathogens of plants of importance to agriculture, forestry and natural ecosystems. Just a few of the most destructive fungal pathogens include the rusts and smuts that affect grain crops, powdery mildews that damage a huge range of crops, the rice blast fungus, and the chestnut blight fungus that eliminated chestnuts from US forests (Van Alfen, N. K. 2001 In: Roberts, K. (ed.), Encyclopedia of Life Science. Wiley InterScience, Chichester.). Fungi also cause serious diseases of immunocompromised humans, such as AIDS patients, leukaemia patients and organ transplant patients. Species causing these human diseases include Candida albicans, Cryptococcus neoformans, Histoplasma capsulatum, Aspergillus fumigatus and Pneumocystis carinii. In addition, Candida albicans, Coccidioides immitus, Paracoccidioides braziliensis, Cryptococcus gattii and several microsporidial fungi can, under some circumstances cause disease on otherwise healthy individuals.

Protozoa cause some of the most deadly and difficult-to-control parasitic diseases of humans and other animals. Apicomplexan parasites include Plasmodium species (which cause malaria in humans and many other animals), Cryptosporidium parvum, Babesia bovis and Toxoplasma gondii. Trypanosomatid parasites include Trypanosoma brucei (sleeping sickness), Trypanosoma cruzi (Chagas disease) and several Leishmania species (leishmaniasis). Amoebic parasites that cause amoebic dysentery include Entamoeba histolytica, Mastigamoeba balamuthi and Giardia species. Trematode (flatworm) parasites include Schistosoma species. Nematode parasites include Onchocerca species (river blindness) and Brugia species (elephantiasis). Nematode parasites also cause many extremely destructive plants diseases, including root knot nematodes (e.g. Meloidogyne species) and cyst nematodes (e.g. Heterodera species).

There is an ongoing need to further characterize pathogenic microorganisms and parasites such as oomycetes in order to provide agents and methods which can be used to treat or prevent the infections they cause.

### SUMMARY OF THE INVENTION

Herein is described the discovery that substances of interest can be selectively delivered to a targeted cell of interest via a cell surface lipid that is characteristic of the cell. This is accomplished by contacting the cell with a fusion construct comprising i) a first domain that binds specifically or selectively to the characteristic cell surface lipid; and ii) a second domain exhibiting an activity of interest. In some embodiments, binding of the first domain to the lipid results in entry, into the cell, of the fusion protein, and hence delivery of the second domain into the cell, where the activity of interest is expressed. In other embodiments, the activity of interest takes place at the cell surface. In either case, the characteristic lipid in effect “recruits” the fusion construct to the cell.

In one embodiment, the cell that is targeted for delivery of a substance of interest is pathogenic or is part of a pathogenic organism. As described herein, characteristic lipids on the surface of one or more cells of a pathogenic organism function as gateways for the specific or selective sequestering, on or in the cell, of a domain that is toxic or inhibitory to the pathogen. The ability to specifically or selectively transport one or more of such domains (agents) to a pathogenic cell or organism via binding to a characteristic lipid opens the way for preventing and/or treating diseases and conditions caused by these pathogens.

In an exemplary embodiment, it has been discovered that the hyphae of oomycetes carry phosphatidylinositol-4-phosphate (PI-4-P) on their outer surface, whereas plant and animal cells do not. It is thus possible to selectively target oomycetes by using molecules which contain a PI-4-P binding domain, and at least one additional domain that exhibits an activity of interest. For example, the additional domain may be toxic, damaging and/or inhibitory or otherwise detrimental to oomycetes. Plant and animal host cells are advantageously immune to the toxicity, damage or inhibition, since they do not have PI-4-P on their surfaces, and thus the construct does not bind to them.

In other embodiments, the cells that are targeted are not pathogenic or are not part of a pathogenic organism, but are targeted for a different reason. For example, using this invention, a characteristic lipid on the surface of a cell can be used to recruit a construct which enhances production of a substance of interest or which promotes an activity of interest in a recombinant or native cell.

The invention also generally provides fusion constructs comprising domains which bind a characteristic lipid on the surface of a cell of interest, and domains which mediate a desired effect on the cell of interest. In further embodiments, the invention provides plant or animal cells that are genetically engineered to produce substances (e.g. proteins) which interfere with the normal functioning of one or more characteristic lipids.

It is an object of this invention to provide a fusion construct comprising at least one first domain specific or selective for binding to a characteristic lipid on the surface of a cell; and at least one second domain with an activity of interest. In some embodiments, the cell is a pathogen or other symbiont. Exemplary pathogens and symbionts include but are not limited to: an archaebacterium, a bacterium, a fungus, an oomycete, an apicomplexan parasite, a trypanosomatid parasite, an amoebozoan parasite, a nematode parasite, a trematode parasite, a microsporidial parasite, an algal parasite, an animal parasite, a plant parasite, Phytophthora, Pythium, downy mildew, Peronospora, Sclerospora, Peronosclerospora, Sclerophthora, Albugo, Aphanomyces, Saprolegnia, Achlya, Puccinia, Phakopsora, Phoma, Ascochyta, Cryphonectria, Magnaporthe, Gaeumannomyces, Synchytrium, Ustilago, Tilletia, Erysiphe, Blumeria, Alternaria, Botrytis, Diaporthe, Fusarium, Leptosphaeria, Macrophomina, Monilinia, Mycosphaerella, Phialophora, Phymatotrichopsis, Taphrina, Aspergillus, Verticillium, Septoria, Pyrenophora, Colletotrichum, Sclerotinia, Sclerotium, Thielaviopsis, Coccidioides, Paracoccidioides, Pneumocystis, Histoplasma, Cryptococcus, Candida, Plasmodium, Babesia, Cryptosporidium, Toxoplasma, Trypanosoma, Leishmania, Entamoeba, Mastigamoeba, Schistosoma, Onchocerca, Giardia, Enterocytozoon, Encephalitozoon, Glomus, Gigaspora, Acaulospora, Tuber, Trichoderma, Epichloe, Neotyphodium, Taxomyces, Nodulisporium, Triphysaria, Striga, and Cuscuta, etc.

In other embodiments, the cell displaying a characteristic lipid is a cancer cell or other pathological cell, including but not limited to a cell infected by a kind of pathogen that requires the host cell to remain alive in order to persist, reproduce, proliferate or spread.

In one embodiment of the invention, the pathogen is an oomycete, the characteristic lipid is phosphatidylinositol-4-phosphate (PI-4-P); the at least one first domain comprises a protein or polypeptide specific or selective for binding to PI-4-P; and the at least one second domain is toxic or inhibitory to the oomycete.

In some embodiments, the characteristic lipid is selected from the group consisting of proteolipids, glycolipids, sphingolipids, phospholipids, sulfolipids and sterols. Exemplary characteristic lipid include but are not limited to phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), lysophosphatidyl-inositol-3,4,5-triphosphate (LPI-3,4,5-P3), phosphatidyl-inositol (PI), lysophosphatidyl-inositol (LPI); phosphatidyl-serine (PS), phosphatidyl-glycerol (PG), phosphatidyl-ethanolamine (PE), phosphatidyl-choline (PC), lysophosphatidyl-serine (LPS), lysophosphatidyl-glycerol (LPG), lysophosphatidyl-ethanolamine (LPE), lysophosphatidyl-choline (LPC), phosphatidic acid (PA), lysophosphatidic acid (LPA), sphingosine-1-phosphate (S-1-P), ceramide-1-phosphate (C-1-P), a glycosylphosphatidylinositol (GPI)-protein anchor, a galactolipid, a glycoceramide, glucosyl-ceramide, galacto-ceramide, glycosylsphingosylinositol (GSI), glycosyl phosphoryl inositol ceramide (GPIC), sphingomyelin (SM), and ergosterol.

In some embodiments of the invention, the at least one first domain comprises a moiety such as: a pleckstrin homology (PH) domain, a protein kinase C domain 1 homology (C1) domain, a protein kinase C domain 2 homology (C2) domain, a Fab 1, YOTB, Vac 1 and EEA1 homology (FYVE) domain, a Phagocytic oxidase homology (PX) domain, an Epsin N terminal Homology (ENTH) domain, a Bin-Amphiphysin-Rvs (BAR) domain, a Four point one protein, Ezrin, Radixin and Moesin homology (FERM) domain, a post synaptic density 95 protein, Drosophila disc large tumor suppressor A, and zonula occludens 1 homology (PDZ) domain, a tubby protein homology (tubby) domain, a defensin, a cathelicidin, a lipid transfer protein. In other embodiments, the at least one first domain comprises a moiety selected from the group consisting of: human phosphatidylinositol-4-phosphate adaptor protein-1 (FAPP1) PH domain, a human phosphatidylinositol-3-phosphate-binding PH-domain protein-1 (PEPP1)-PH domain, an Arabidopsis-PH-domain-protein-1 (AtPH1) PH domain, a soybean AtPH1-homolog (GmPH1) PH domain, an Arabidopsis Enhanced Disease Resistant-2 (EDR2) PH domain, an Arabidopsis phosphatidylinositol-4-kinase (PI4K) PH domain, a potato EDR2 PH domain, a tobacco PI4K PH domain, a soybean EDR2 PH domain, a soybean PI4K PH domain, Raphanus sativus Anti-Fungal Peptide-2 RsAFP2, Dahlia merckii Anti-Microbial Peptide (DmAMP1), and defensin Bombyx mori cecropin B.

In some embodiments, the at least one second domain with an activity of interest binds to or covalently modifies a protein of the cell. In other embodiments, the at least one second domain with an activity of interest binds to or covalently modifies a nucleic acid of the cell. In yet other embodiments, the at least one second domain with an activity of interest binds to or covalently modifies a lipid of the cell. In further embodiments, the at least one second domain with an activity of interest binds to or covalently modifies a carbohydrate of the cell. In other embodiments, the at least one second domain with an activity of interest binds to or covalently modifies a small molecule within the cell. Exemplary “small molecules” include but are not limited to adenosine triphosphate (ATP), nicotinamide adenine dinucleotide (NAD), an amino acid, and a nucleotide triphosphate.

In other embodiments, the invention provides methods of delivering a substance of interest to a cell. The methods comprise the step of contacting the cell with a fusion construct comprising 1) at least one first domain specific or selective for binding to a characteristic lipid on the surface of said cell; and 2) at least one second domain comprising the substance of interest. In some embodiments, the at least one second domain comprising the substance of interest is capable of modifying the metabolism, physiology, development or growth of the cell.

In some embodiments, the invention provides methods of killing, damaging or inhibiting a pathogenic cell. The methods comprise the step of contacting the cell with a fusion construct comprising: 1) at least one first domain specific or selective for binding to a characteristic lipid on the surface of the pathogenic cell; and 2) at least one second domain capable of killing, damaging or inhibiting the pathogenic cell.

In other embodiments, the invention provides methods of delivering a substance of interest to a target cell, the target cell being located within a host cell. The methods comprise the step of contacting the host cell that contains the target cell with a fusion construct comprising 1) at least one domain specific or selective for binding to or interacting with a proteins or lipid on the surface of the host cell. In particular embodiments, the domain may be specific for a characteristic lipid on the surface of the host cell; and 2) at least one first domain specific or selective for binding to a characteristic lipid on the surface of the target cell; and 3) at least one second domain comprising said substance of interest.

The invention further provides methods of killing, damaging or inhibiting a pathogenic cell that is contained within a host cell. The methods comprise the step of: contacting the host cell containing the pathogenic cell with a fusion construct which comprises 1) at least one domain specific or selective for binding or interacting with a protein or lipid on the surface of the host cell. In particular embodiments, the domain may be specific for a characteristic lipid on the surface of the host cell; 2) at least one first domain specific or selective for binding to a characteristic lipid on the surface of the pathogenic cell; and at least one second domain capable of killing, damaging or inhibiting the pathogenic cell, e.g. by preventing reproduction of the pathogen and/or by curtailing the spread of infection.

In one embodiment, the invention provides plant, animal or microbial cells that are to genetically modified to contain and express nucleic acid sequences encoding a protein construct comprising 1) at least one first domain specific or selective for binding to a characteristic lipid on the surface of a target cell; and 2) at least one second domain with an activity of interest. In some embodiments, the target cell is a microbial cell. In other embodiments, the microbial cell is a symbiotic microbial cell and the plant, animal or microbe is a host of the symbiotic microbial cell. In various embodiments, the symbiotic microbial cell may be mutualistic with, commensal on, or pathogenic to said host plant, animal or microbe.

In some embodiments, the at least one second domain with an activity of interest alters the metabolism, physiology, development or growth of the symbiotic microbial cell. In yet other embodiments, the symbiotic microbial cell is pathogenic and the at least one second domain is capable of killing, damaging or inhibiting the symbiotic microbial cell.

The invention also provides methods of killing or inhibiting a pathogen by interfering with the activity of a characteristic lipid on a cell surface of the pathogen. The methods comprise the step of contacting the pathogen with a single domain agent which binds to and interferes with the activity of the characteristic lipid, wherein interference kills or inhibits the pathogen. In exemplary embodiments, the pathogen is an oomycete and the characteristic lipid is phosphatidylinositol-4-phosphate (PI-4-P). In further exemplary embodiments, the agent is a phosphotidylinositol-specific phospholipase C. Plant, animal and microbial cells that are genetically modified to contain and express nucleic acid sequences encoding a protein that specifically or selectively binds to a characteristic lipid on the surface of a pathogen and exhibits an activity which interferes with a function of the characteristic lipid, are also provided. Interference with the functioning of the characteristic lipid kills, inhibits or otherwise damages the pathogen, e.g. by killing outright, by preventing reproduction and thus the spread of infection, etc. Thus, such genetically modified cells are protected from infection by the pathogen, or from the development of symptoms associated with infection by the pathogen.

### BRIEF DESCRIPTION OF THE DRAWINGS

FIG. 1. Identification of characteristic lipids on the surface of Phytophthora sojae hyphae, soybean root cells and human A549 lung epithelial cells.

A, Phosphatidylinositol-4-phosphate (PI-4-P) on the surface of P. sojae membranes mediates selective protein entry. Phosphatidylinositol-4-phosphate adaptor protein-1 (FAPP1)-mCherry, FAPP1-GFP, mCHerry alone or phosphatidylinositol-3-phosphate-binding PH-domain protein-1 (PEPP1)-GFP proteins (1 mg/ml) were incubated together with P. sojae hyphae for 12 hr, then washed extensively. Left panels show fluorescence image; right panels show the matching light micrograph.

B, Detection of PI-3-P but not PI-4-P on the surface of soybean cells. PEPP1-GFP or FAPP1-GFP proteins (1 mg/ml in 25 mM MES pH 5.8) were incubated with soybean root suspension culture cells for 6 hr or with soybean root tips for 12 hr at 4° C. then washed for 2 hr with 25 mM MES pH 5.8, then plasmolyzed with 0.8M Mannitol (suspension culture cells) or 4M NaCl (roots) for 30 minutes, before being photographed. Left panels show fluorescence image; right panels show the matching light micrograph. Plasma membranes are indicated by the white arrow heads.

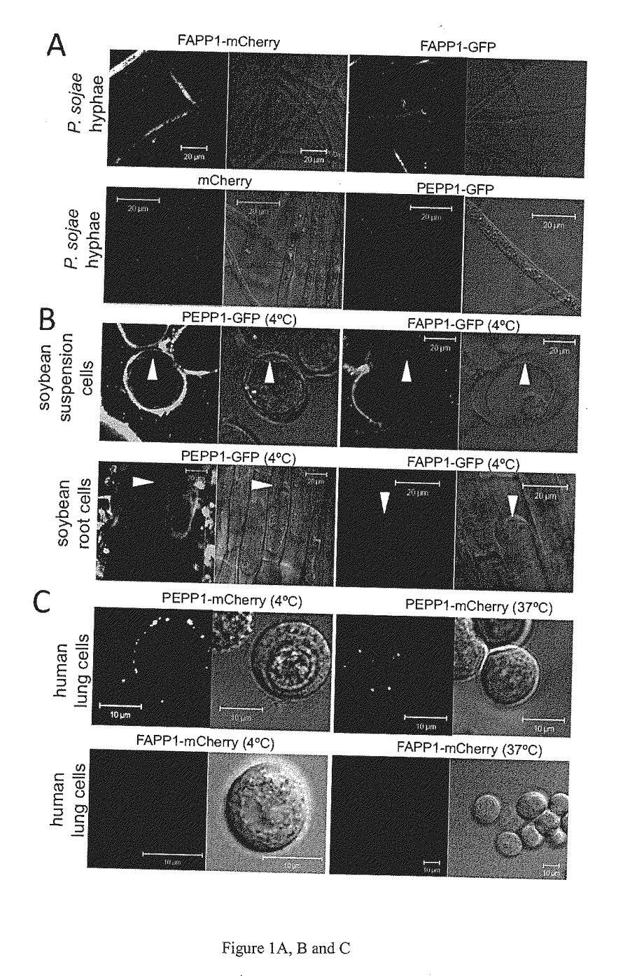

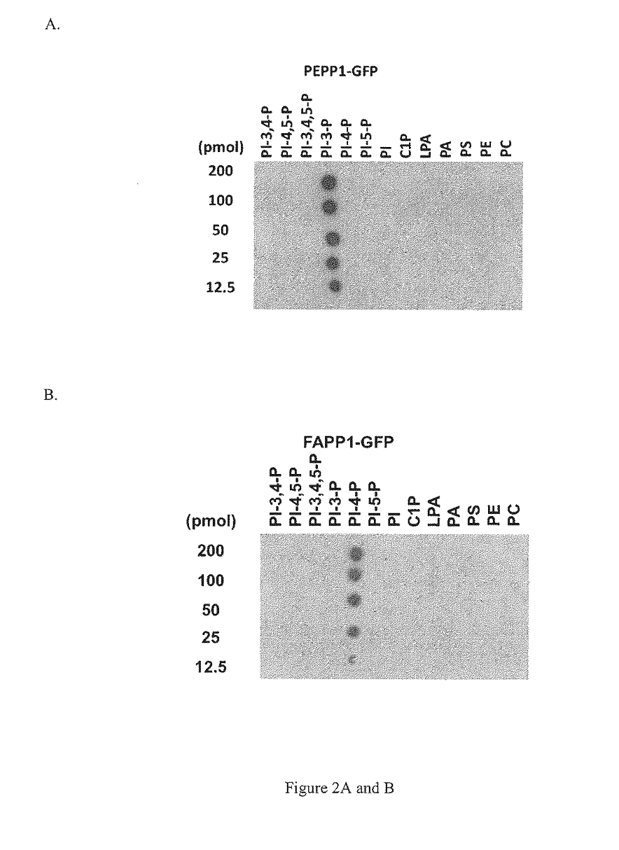

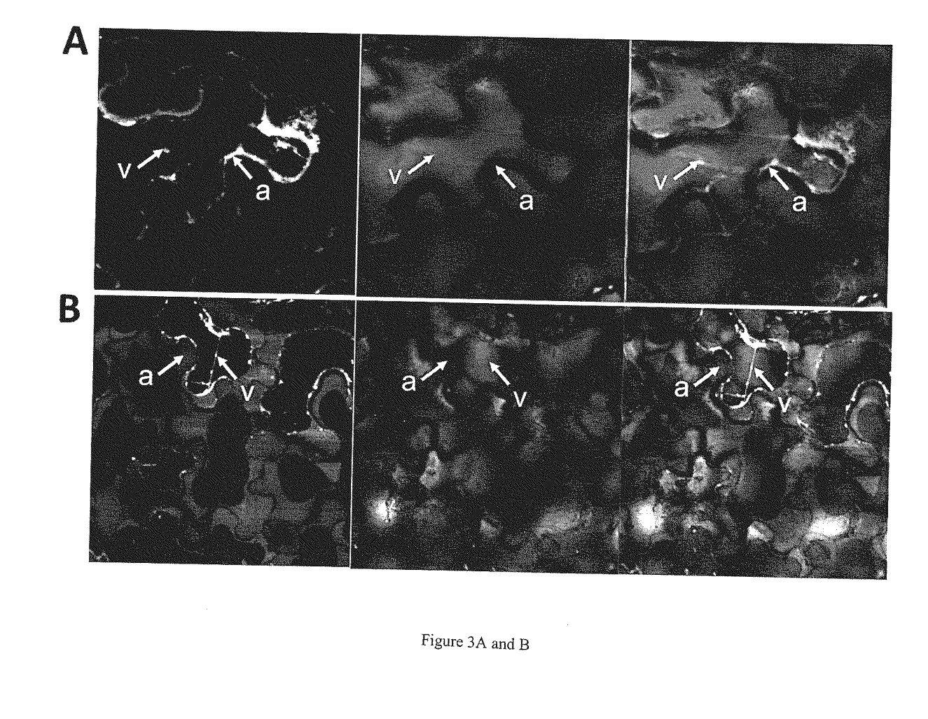

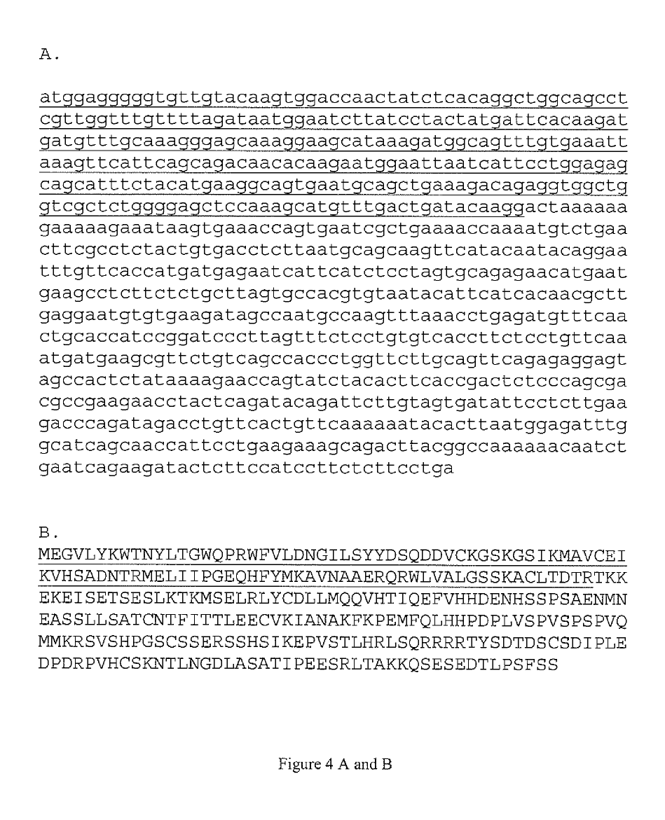

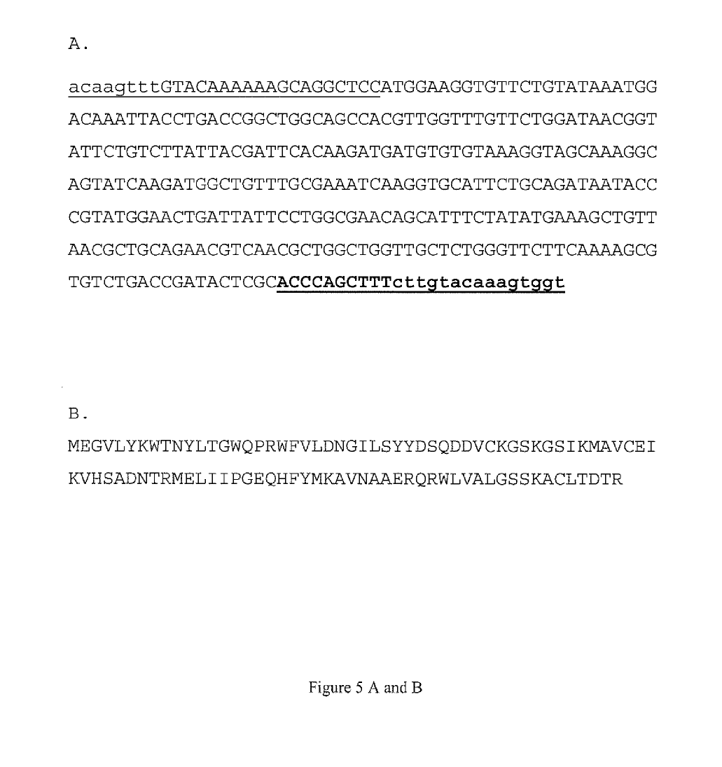

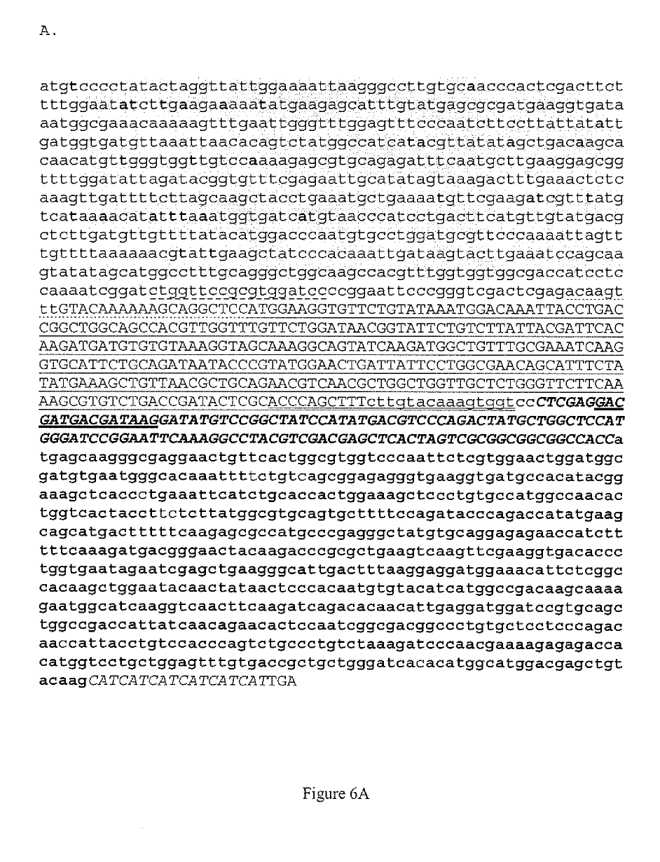

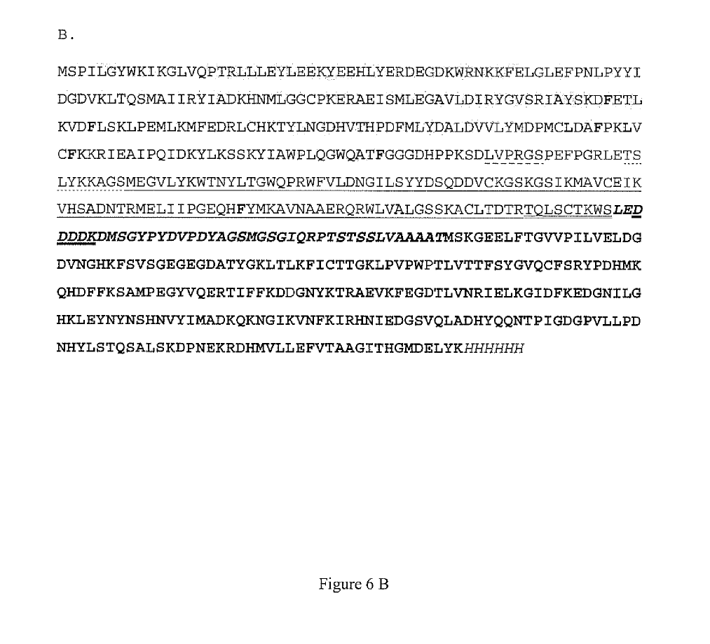

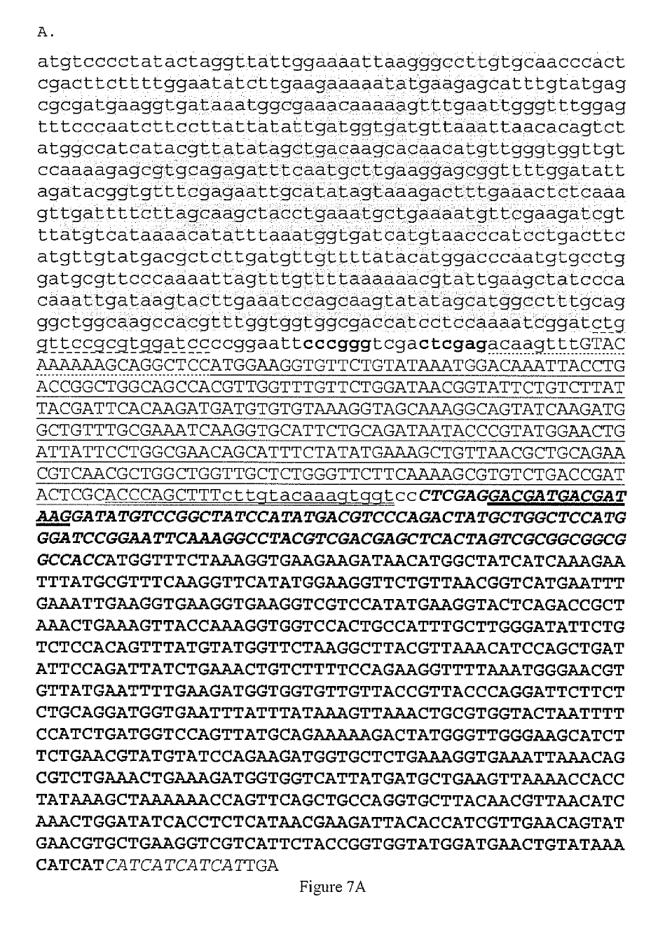

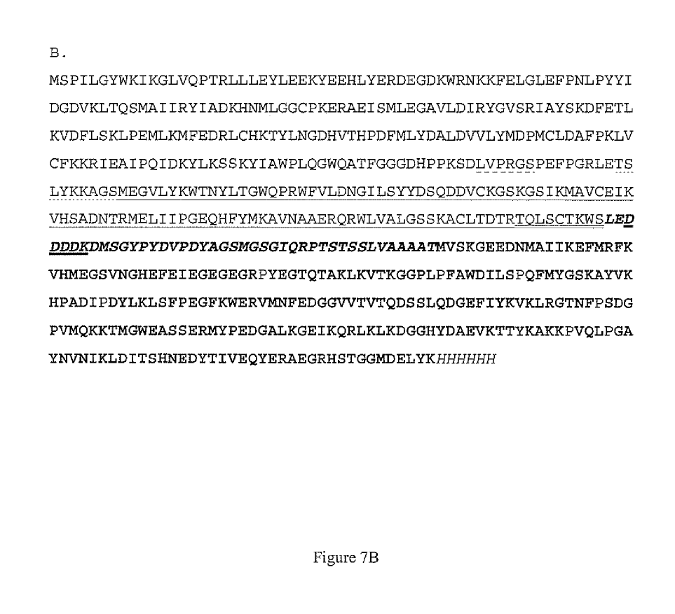

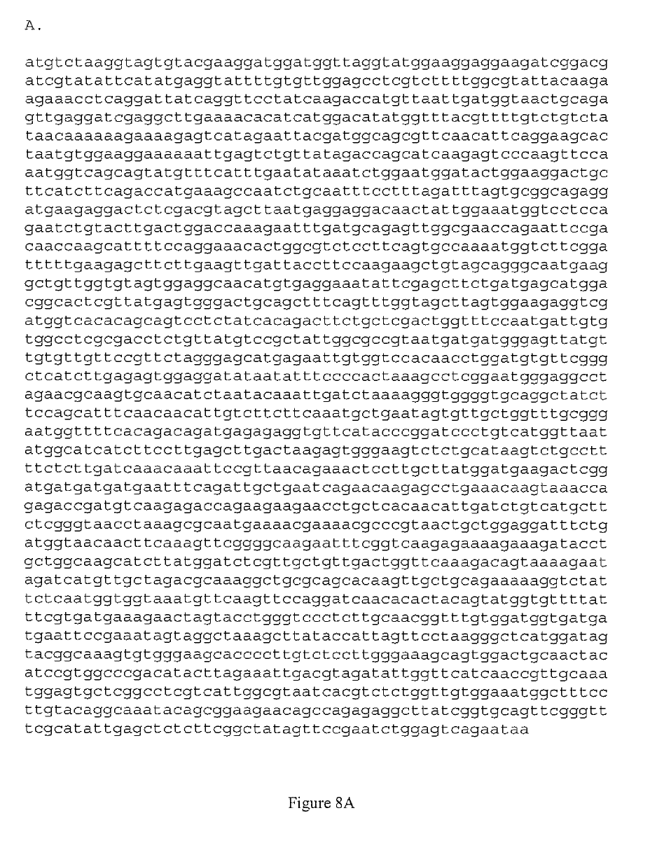

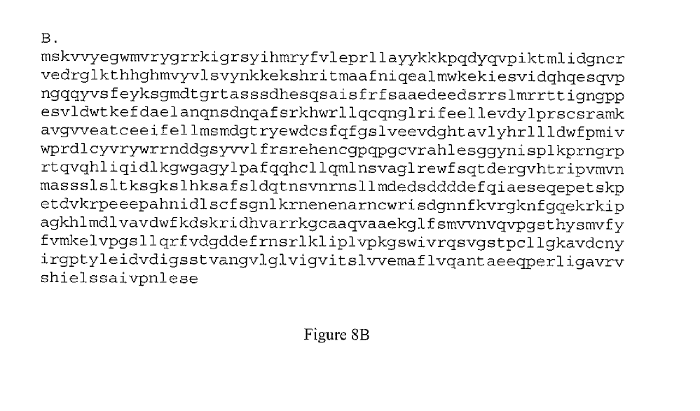

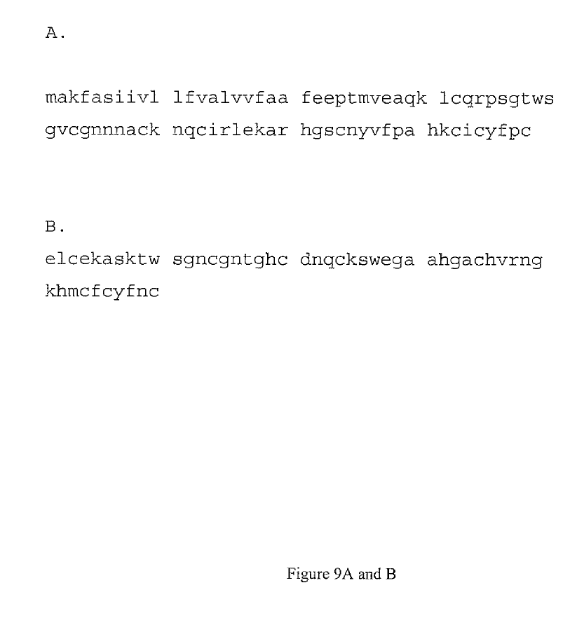

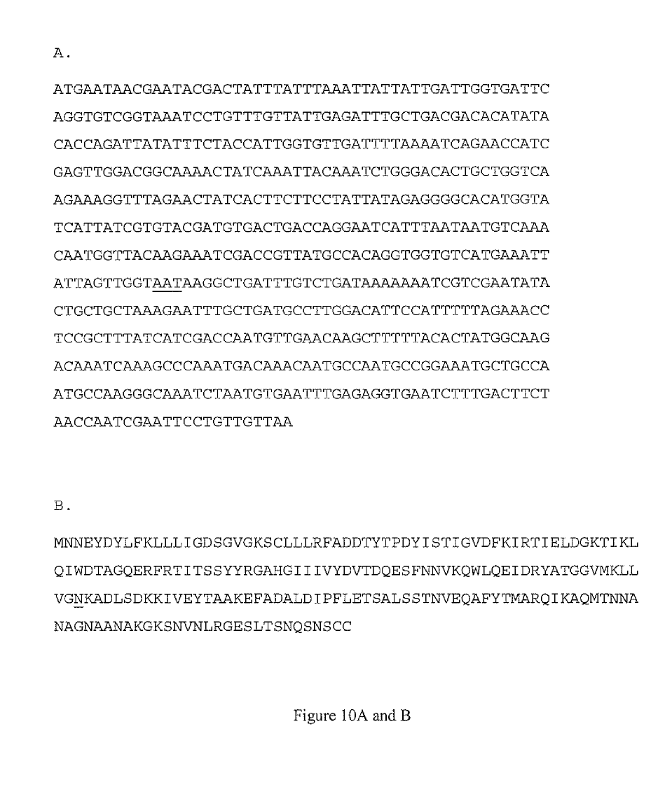

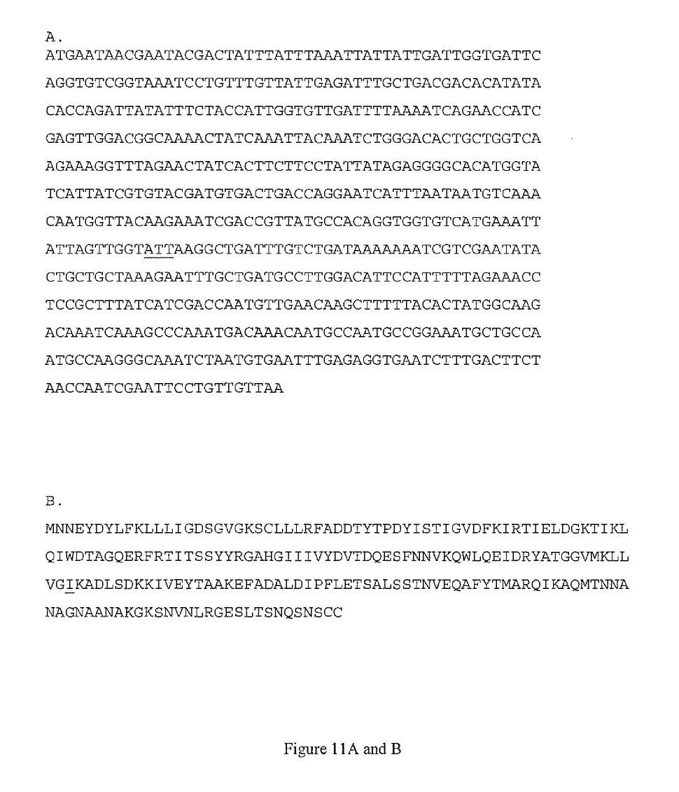

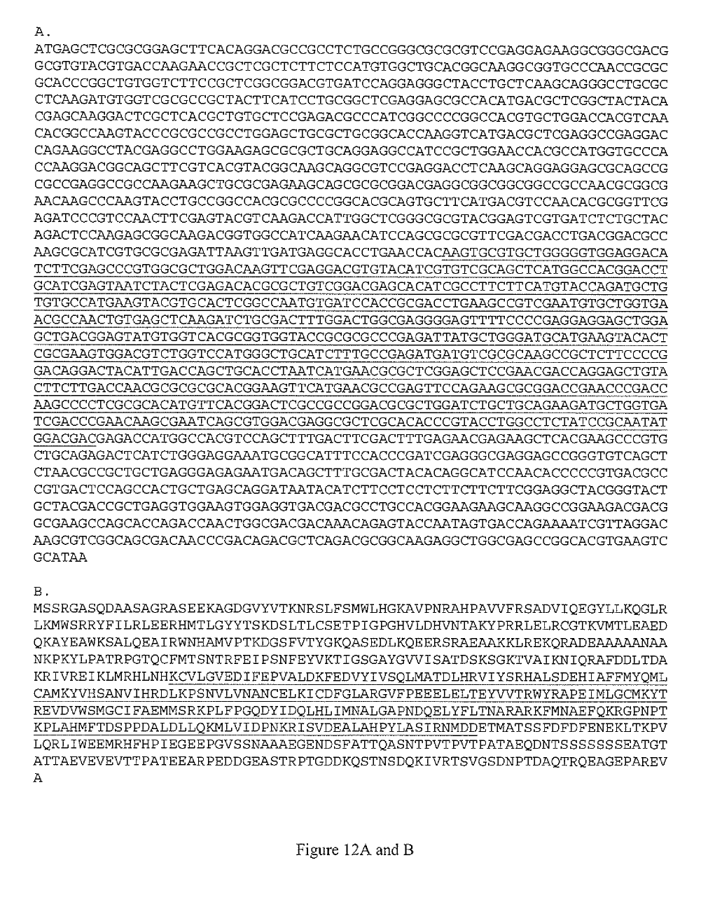

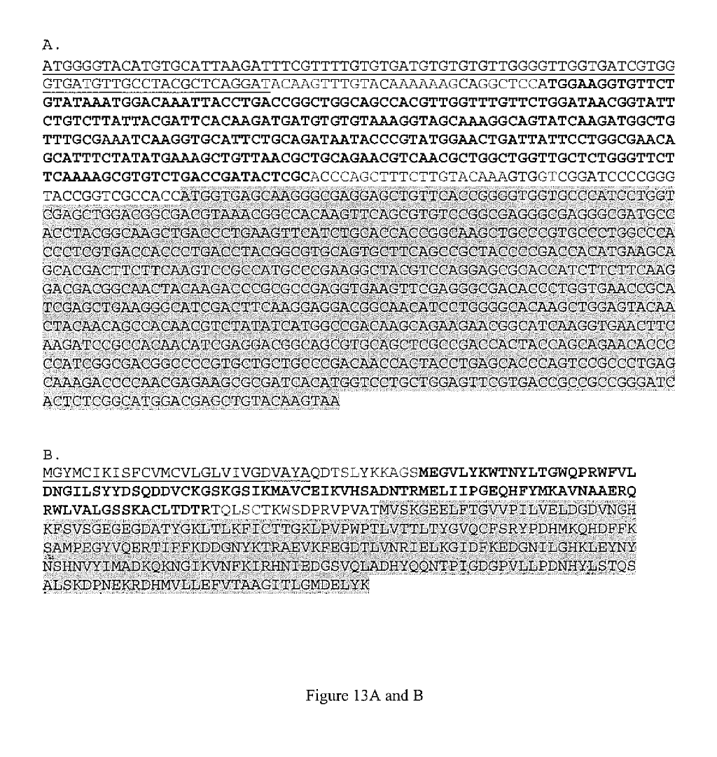

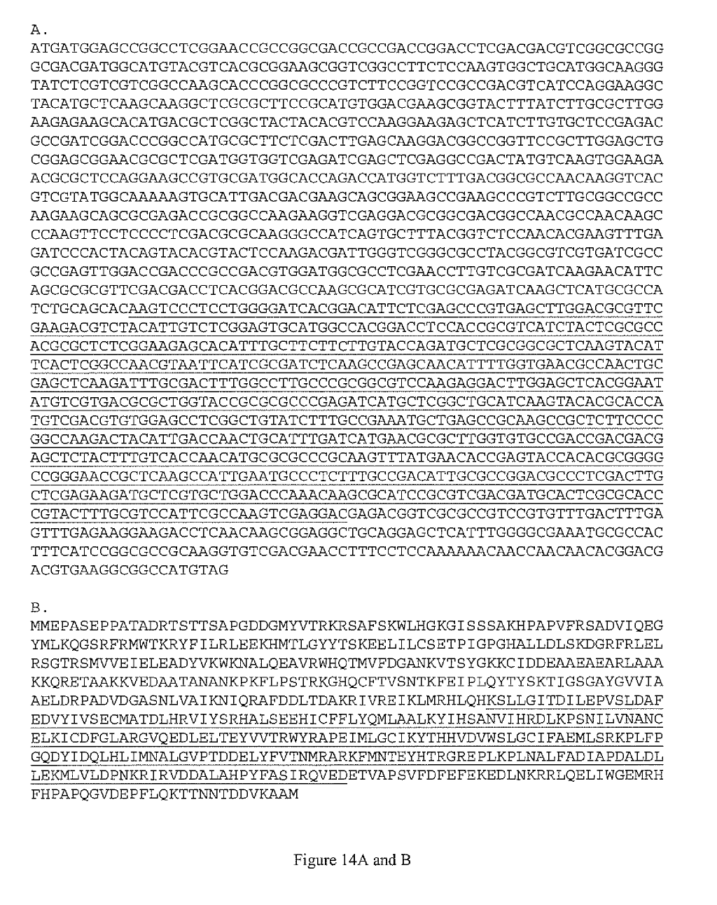

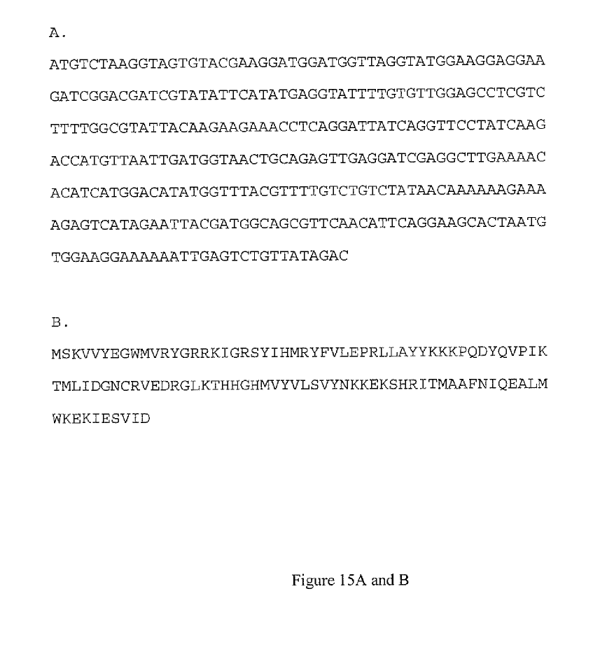

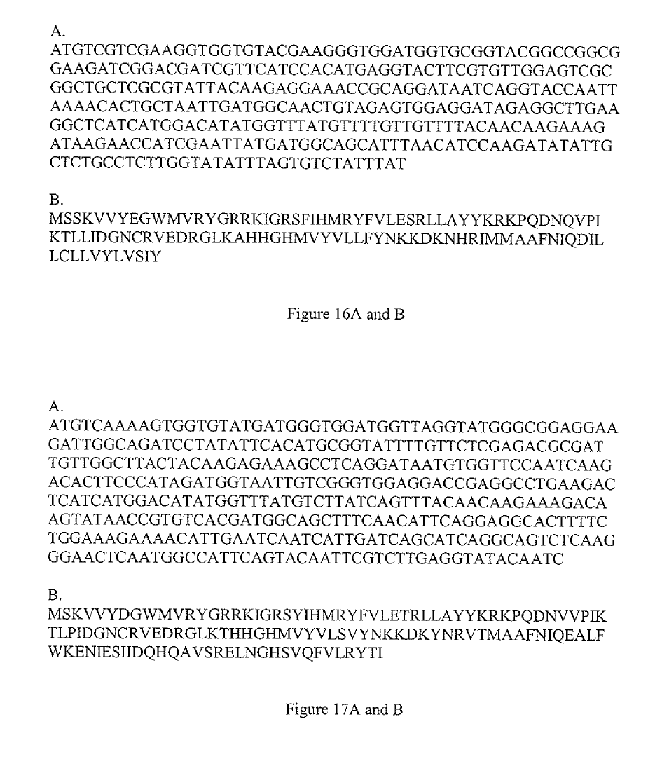

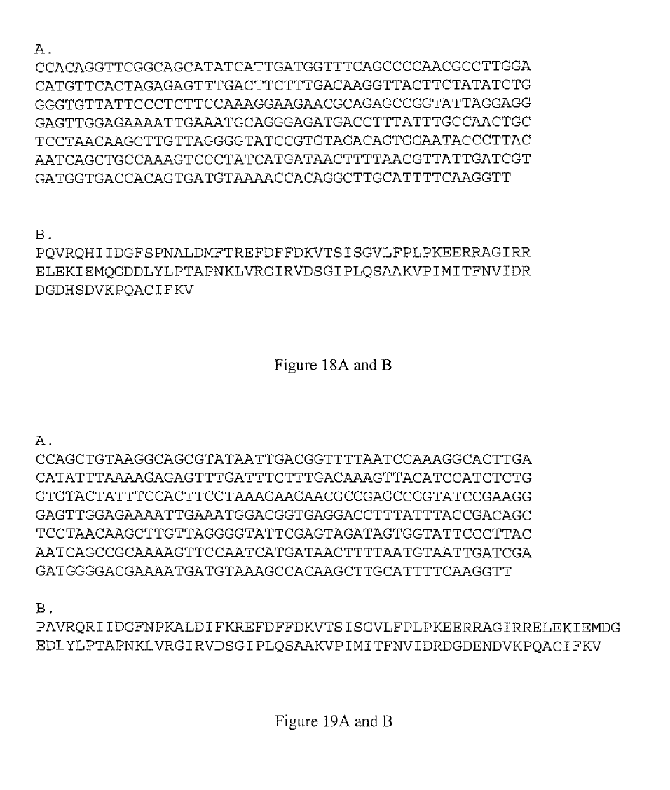

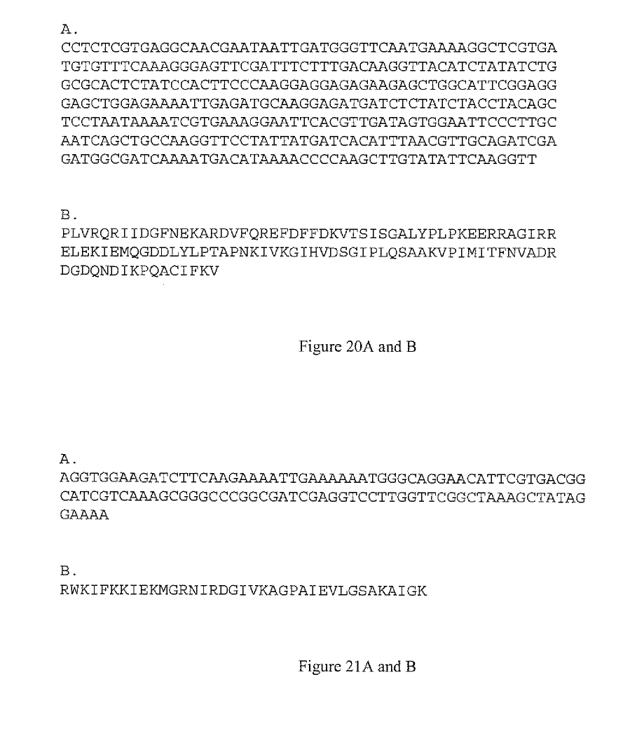

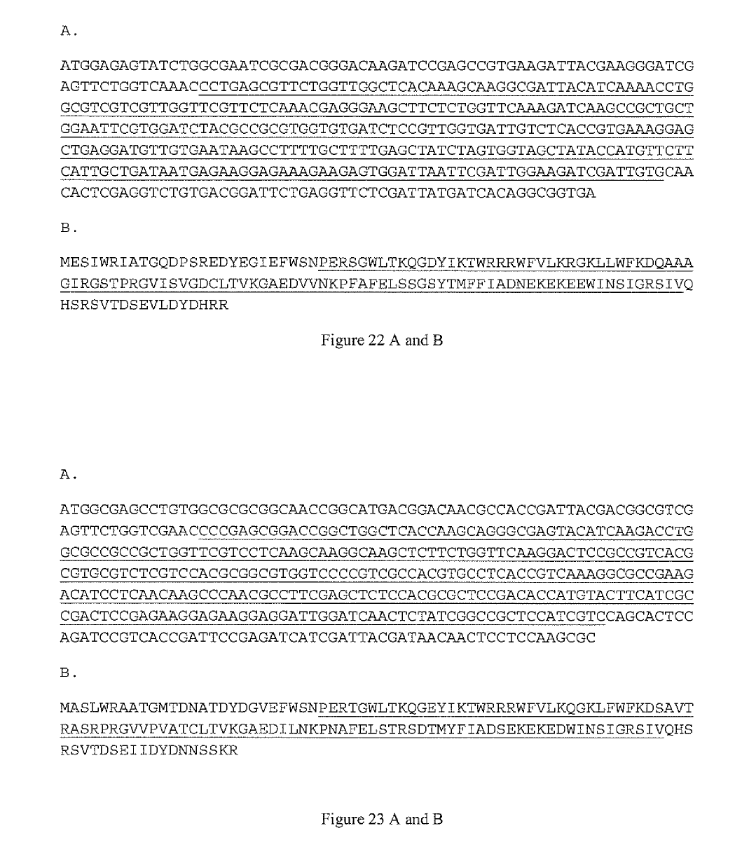

C, Detection of PI-3-P but not PI-4-P on the surface of human lung cells PEPP1-mCherry or FAPP1-mCherry proteins [1 mg/ml in Dulbecco's Phosphate Buffer Saline (Ca⁺²/Mg⁺² free) (DPBS; Gibco)] were incubated with Human lung adenocarcinoma cells A549 (ATCC CCL-185) for 8 hr at 4° C. or 37° C. then washed twice briefly with DPBS, before being photographed. Left panels show fluorescence image; right panels show the matching light micrograph. At 4° C., at which endocytosis is inhibited, PEPP1-mCherry binds to the surface of the cells indicating that PI-3-P is on the surface of the membranes. At 37° C. at which endocytosis is active, PEPP1-mCherry enters inside the cells indicating that PI-3-P-binding enables cell entry. FAPP1-mCherry neither binds to the surface of the cells at 4° C., nor enters the cells at 37° C., indicating that PI-4-P is absent from the surface of the cells, and indicating that cell surface binding is required for cell entry. FIGS. 1B and C were adapted from FIGS. 3 and 5 respectively from Kale et al 2010. External lipid PI-3-P mediates entry of eukaryotic pathogen effectors into plant and animal host cells. Cell 142:284-295.

FIG. 2. Phospholipid-binding specificity of A, PEPP1 and B, FAPP1 biosensors. PEPP1-PH and FAPP1-PH domains were tested for phospholipid binding as fusions with GFP at the C-terminus. Lipid filters were prepared by spotting 1 μl of each lipid at an appropriate series of dilutions onto Hybond-C-extra membranes (GE Healthcare). After blocking of the filter, the respective fusion protein (20 μg) was added and incubated overnight at 4° C. After washing, bound proteins were detected with rabbit anti-GFP antibody followed by peroxidase-conjugated anti-rabbit antibody and ECL reagent. PI-3,4-P=phosphatidylinositol-3,4-bisphosphate; PI-4,5-P=phosphatidylinositol-4,5-bisphosphate; PI-3,4,5-P=phosphatidylinositol-3,4,5-triphosphate; PI-3-P=phosphatidylinositol-3-phosphate; PI-4-P=phosphatidylinositol-4-phosphate; PI-5-P=phosphatidylinositol-5-phosphate; PI=phosphatidylinositol; C1P=ceramide-1-phosphate; LPA=lysophosphatidic acid; PA=phosphatidic acid; PS=phosphatidylserine; PE=phosphatidylethanolamine; PC=phosphatidylcholine.

FIG. 3. Secretion of a PI-4-P-binding fusion protein, FAPP1-GFP from plant cells. Nicotiana benthamiana leaves were infiltrated with Agrobacterium tumefaciens cells that delivered a transfer-DNA (T-DNA) into the plant cells that encoded a protein consisting of a fusion between a secretory leader, the PI-4-P-binding protein FAPP1, and green fluorescent protein (GFP). After 12 hours, cells from the infiltrated leaf area were examined by confocal microscopy, without (A) or with (B) plasmolysis treatment (30 min exposure to 0.8 M mannitol). Panels show fluorescence image (left), light image (middle), merged image (right). FAPP1-GFP fusion protein can clearly be seen to be accumulating in the apoplast (a). Secretory vesicles (v) can also be seen within the cells in both A and B.

FIGS. 4A and B. Full length naturally occurring FAPP1 protein. A, nucleotide sequence (SEQ ID NO: 1); B, amino acid sequence (SEQ ID NO: 2); the lipid binding domain is underlined.

FIGS. 5A and B. Synthetic FAPP1 including attB sites used for Gateway homologous recombination cloning. A, nucleotide sequence (SEQ ID NO: 3); B, amino acid sequence (SEQ ID NO: 4); attB1 is underlined; attB2 is underlined and in bold.

FIGS. 6A and B. GST-FAPP1-GFP. A, nucleotide Sequences as present in pSDK1 vector (SEQ ID NO: 5); B, amino acid sequence (SEQ ID NO: 6). In both sequences: light grey shading=glutathione-S-transferase (GST); dashed underline=thrombin cleavage site; dotted underline=AttB1 site; underline=FAPP1; double underline=AttB2 site; bold italics=linker sequence; bold underline within the linker sequence=enterokinase cleavage site; bold=mCherry; italic capitals=His tag.

FIGS. 7A and B. GST-FAPP1-mCherry. A, nucleotide sequences as present in pSDK2 vector (SEQ ID NO: 7); B, amino acid sequence (SEQ ID NO: 8). In both sequences: light grey shading=GST; dashed underline=thrombin cleavage site; dotted underline=AttB1 site; underline=FAPP1; double underline=AttB2 site; bold italics=linker sequence; bold underline within the linker sequence=enterokinase cleavage site; bold=GFP; italic capitals=His tag.

FIGS. 8A and B. Arabidopsis EDR2 A, nucleotide sequence (SEQ ID NO: 9); B, amino acid sequence (SEQ ID NO: 10).

FIGS. 9A and B. A, Radish AFP2 peptide (GenBank P30230.4 GI:1703206, SEQ ID NO: 11); and B, Dahlia defensin peptide DmAMP1 (GenBank P0C8Y4.1 GI:229890071, SEQ ID NO: 12).

FIGS. 9A and B. Candida albicans YPT1, A, nucleic acid sequence (SEQ ID NO: 13) and B, amino acid sequence (SEQ ID NO: 14).

FIGS. 11A and B. Candida albicans YPT1(N121I). A, nucleic acid sequence (SEQ ID NO: 15) and B, amino acid sequence (SEQ ID NO: 16). The mutation site is underlined in both A and B.

FIGS. 12A and B. Phytophthora sojae PsSAK1. A, DNA sequence (region encoding KIM docking site is underlined, SEQ ID NO: 17) and B, amino acid sequence (SEQ ID NO: 18, KIM docking site of PsSAK1 located between amino acids 296 and 539, is underlined).

FIGS. 13A and B. A, DNA sequence encoding signal peptide (SP)-FAPP1-GFP construct (SEQ ID NO: 19); B, amino acid sequence of SP-FAPP1-GFP construct (SEQ ID NO: 20). For both sequences: underlined=PR1a signal peptide; grey=AttB gateway recombination sequences; bold=PH domain of FAPP1; highlighted grey=eGFP sequence.

FIGS. 14A and B. Saprolegnia parasitica SpSAK1 A, DNA sequence (region encoding KIM docking site is underlined) (SEQ ID NO: 21) and B, amino acid sequence (SEQ ID NO: 22); KIM docking site of SpSAK1 located between amino acids 303 and 544, is underlined).

FIGS. 15 A and B. Arabadopsis thaliana EDR2 PH domain A, DNA sequence (SEQ ID NO: 23); B, amino acid sequence (SEQ ID NO: 24).

FIGS. 16 A and B. Soybean EDR2 pleckstrin homology (PH) domain. A, DNA sequence (SEQ ID NO: 25); B, amino acid sequence (SEQ ID NO: 26).

FIGS. 17A and B. Potato EDR2 pleckstrin homology (PH) domain. A, DNA sequence (SEQ ID NO: 27); B, amino acid sequence (SEQ ID NO: 28).

FIGS. 18A and B. Arabidopsis phosphatidylinositol-4-kinase PH domain. A, DNA sequence (SEQ ID NO: 29) and amino acid sequence (SEQ ID NO: 30).

FIGS. 19A and B. Soybean phosphatidylinositol-4-kinase PH domain. A, DNA sequence (SEQ ID NO: 31) and amino acid sequence (SEQ ID NO: 32).

FIGS. 20A and B. Tobacco phosphatidylinositol-4-kinase PH domain. A, DNA sequence (SEQ ID NO: 33) and amino acid sequence (SEQ ID NO: 34).

FIGS. 21A and B. Silkworm (Bombyx mori) cecropin B mature peptide. A, DNA sequence (SEQ ID NO: 35) and amino acid sequence (SEQ ID NO: 36).

FIGS. 22A and B. Arabidopsis-PH-domain-protein-1 (AtPH1) PH domain, A, nucleic acid (SEQ ID NO: 37); B, amino acid (SEQ ID NO: 38). Underline=PH domain of PH1.

FIGS. 23A and B. Exemplary soybean AtPH1-homolog (GmPH1) PH domain. A, nucleic acid (SEQ ID NO: 39); B, amino acid (SEQ ID NO: 40). Underline=PH domain of PH1.

### DETAILED DESCRIPTION

The invention provides compositions and methods for the targeted delivery of a substance with an activity of interest to a cell. The compositions and methods take advantage of the discovery that characteristic cell surface lipids can be used to mediate the transport of a substance of interest to and/or into a cell. In particular, the invention provides constructs comprising at least one first domain that binds specifically or selectively to a cell via a cell surface lipid that is characteristic of the cell, and at least one second domain that exhibits or carries out an activity of interest that affects the cell. In some embodiments, after binding to the cell, the construct or at least the second domain of the construct is taken up by the cell (e.g. via endocytosis) and the second domain carries out its activity within the cell. In other embodiments, the construct remains on the cell surface and the second domain carries out its activity on the cell surface.

By “domain” we mean a moiety or portion of a construct (e.g. a protein, polypeptide, peptide, small molecule, etc. as described herein) the activity of which is generally distinct separable from that of other domains of the construct. Domains may be physically separable from one another and still retain their activity, and/or may have distinct origins (originally obtained from different species, or from different proteins, etc.). As described herein, first and second domains may be “mixed and matched” i.e. a particular first domain may be selected for its function (binding to a characteristic lipid) and may be coupled or attached to any one (or in some embodiments, more than one) second domain to impart the activity of the second domain to the construct.

### Characteristic Cell Surface Lipids

By “characteristic” cell surface lipids we mean a type of lipid molecule, at least a bindable portion of which is present (or exposed or accessible) at or on the surface of only one type of cell, or at most on only a few types of cells, or at least only one or a few types of cells in a given environment. For example, the lipid may occur on the cells of several or even many different species within a genus of organisms, but the cells that are targeted as described herein may be present only in a particular environment under consideration, e.g. the interior of a mammalian body that is being treated, the habitat of a particular plant that is being treated, etc. In such circumscribed environments, the cells that are targeted are the only cells with the characteristic lipid present on their surface, and the lipids are thus “characteristic” of those cells in that environment. In some environments, more than one type of cell possessing a particular characteristic lipid may be targeted, e.g. oomycetes and fungi may both be targeted by a single construct if they share a common characteristic lipid. Generally, the abundance of the characteristic lipid on the cell surface of targeted cells will be at least 10 fold or more (e.g. 50, 100, 500, or even 1000 fold or more) than on the surfaces of cells that are not targeted. In some embodiments, a particular “type” of cell refers to single cells or cells which are part of an organism of a particular group, e.g. phylum, class, order, family, genus, species, clad, or other phylogenetic classification, e.g. oomycetes, fungi, apicomplexans, trypanosomatids, nematodes, trematodes, amebozoans, etc. A single cell type may have more than one characteristic lipid that is suitable for targeting as described herein (e.g. Takahashi H K et al. 2009. Current relevance of fungal and trypanosomatid glycolipids and sphingolipids: studies defining structures conspicuously absent in mammals. Ann Acad Bras Cienc 81:477-488).

A characteristic lipid may be identified through the use of specific lipid binding proteins that have been fused or attached to a detectable label or a detectable moiety such as a fluorescent protein (e.g. green fluorescent protein [GFP] or mCherry fluorescent protein) so that binding and/or entry of the detectable moiety can be measured or observed, e.g. by confocal microscopy. In some cases, especially when a cell has a cell wall, it maybe easier to observe entry of a detectable moiety into the cell (which usually can be observed at a physiological temperature such as 25° C. or 37° C.) than to observe binding of a detectable moiety to the cell surface (which usually can be observed at 0-4° C. when endocytosis is inhibited). In the context of this invention, it is actually more important to observe that the detectable moiety can be internalized in a specific manner, than to observe binding to the surface. If the detectable moiety binds to the surface of one kind of cell (e.g. of an oomycete) or can enter the cell, but cannot bind to or enter a second kind of cell (e.g. a plant cell), then the lipid may be considered characteristic of the first kind of cell (e.g. the oomycete) in the context of an interaction between the two cells (e.g. an oomycete-plant interaction). FIGS. 1 A and B illustrate an example in which phosphatidylinositol-4-phosphate (PI-4-P) was identified as characteristic of the oomycete Phytophthora sojae in the context of the P. sojae-soybean interaction. The PI-4-P-specific protein domain FAPP1-PH was fused to mCherry or GFP while the PI-3-P-specific protein domain PEPP1 was fused to GFP. Both FAPP1 fusion proteins entered P. sojae hyphae at 25° C., but mCherry alone did not (FIG. 1A). On the other hand the FAPP1-GFP fusion did not bind to the surface of soybean cells nor enter them (FIG. 1B). The same experiment identified phosphatidylinositol-3-phosphate as characteristic of soybean in the same interaction. The PEPP1-GFP fusion protein did bind to and partially enter the soybean cells at 4° C. (FIG. 1B) but did not enter the P. sojae hyphae at 25° C. (FIG. 1A). FIG. 1D shows that the PI-3-P-binding fusion protein PEPP1-mCherry binds to human cells at 4° C. and enters them at 37° C. but the FAPP1-mCherry fusion protein does neither. Thus PI-3-P is inferred to occur on the surface of human cells whereas PI-4-P is inferred to be absent. Thus PI-4-P could be considered characteristic of human cells in an interaction with an oomycete such as P. sojae.

Specific lipid binding domains that can be used for these experiments may be derived from naturally occurring proteins such as those listed in Table 1 (Dowler S, et al. 2000 Identification of pleckstrin-homology-domain-containing proteins with novel phosphoinositide-binding specificities. The Biochemical journal 351:19-31; Lemmon M A, 2008. Membrane recognition by phospholipid-binding domains. Nature Reviews 9:99-111; Stace C L & Ktistakis N T. 2006. Phosphatidic acid- and phosphatidylserine-binding proteins. Biochimica et Biophysica Acta 1761:913-926; Snook C F, Jones J A, & Hannun Y A (2006) Sphingolipid-binding proteins. Biochimica et Biophysica Acta 1761:927-946; Sandvig, K. et al. 2010. Protein toxins from plants and bacteria: probes for intracellular transport and tools in medicine. FEBS Len 584, 2626-2634), by selecting random peptides specific for a lipid of interest, or by raising antibodies specific for a lipid of interest (Brown H A. 2007. Lipidomics and Bioactive Lipids: Specialized Analytical Methods and Lipids in Disease. Methods in Enzymology vol 433. Academic Press, San Diego, Calif.).

TABLE 1
A list of some known specific lipid-binding proteins

| | Specific lipid-binding | |
|---|---|---|
| Lipid | protein | References |
| phosphatidylserine | annexin V | Stace C L & Ktistakis N T. 2006. Phosphatidic acid- |
| | Synaptotagmin | and phosphatidylserine-binding proteins. Biochimica |
| | many others | et Biophysica Acta 17618: 913-926 |
| Phosphatidylinositol | MAP2 | Surridge C D & Burns R G 1994 The difference in the |
| | | binding of phosphatidylinositol distinguishes MAP2 |
| | | from MAP2C and Tau. Biochemistry 33(26): 8051-8057. |
| Phosphatidylinositol- | PEPP1-PH | Dowler S, et al. (2000) Identification of pleckstrin- |
| 3-phosphate | Hrs FYVE | homology-domain-containing proteins with novel |
| | VAM7p-PX | phosphoinositide-binding specificities. Biochemical |
| | many others | J. 351: 19-31. |
| Phosphatidylinositol- | FAPP1-PH | Dowler et al. 2000. Biochemical J. 351: 19-31. |
| 4-phosphate | many others | |
| Phosphatidylinositol- | PLCδ1-PH | Dowler et al. 2000. Biochemical J. 351: 19-31. |
| 4,5-diphosphate | many others | |
| Phosphatidylinositol- | TAPP1-PH | Dowler et al. 2000. Biochemical J. 351: 19-31 |
| 3,4-diphosphate | TAPP2-PH | |
| Phosphatidylinositol- | Centaurin-β2-PH | Dowler et al. 2000. Biochemical J. 351: 19-31 |
| 3,5-diphosphate | | |
| Phosphatidylinositol- | Grp1-PH | Dowler et al. 2000. Biochemical J. 351: 19-31 |
| 3,4,5-triphosphate | Evectin-2-PH | |
| Phosphatidic acid | PP 1cγ | Stace C L & Ktistakis N T. 2006. Phosphatidic acid- |
| | Raf-1 | and phosphatidylserine-binding proteins. Biochimica |
| | many others | et Biophysica Acta 17618: 913-926 |
| Diacylglycerol | Protein kinase Cα | Toker A. 2005. The biology and biochemistry of |
| | RasGRP | diacylglycerol signalling. EMBO reports 6: 310-314. |
| | many others | |
| ceramide | ceramide kinase | Snook C F, Jones J A, & Hannun Y A. 2006. |
| | Raf-1 | Sphingolipid-binding proteins. Biochimica et |
| | many others | biophysica acta 1761: 927-946. |
| ceramide-1-phosphate | phospholipase | Snook C F, Jones J A, & Hannun Y A. 2006. |
| | A2α | Biochimica et Biophysica Acta 1761: 927-946. |
| sphingosine | Sphingosine kinases | Snook C F, Jones J A, & Hannun Y A. 2006. |
| | protein kinase C | Biochimica et Biophysica Acta 1761: 927-946, |
| sphingosine-1- | S-1-P phosphatases | Snook C F, Jones J A, & Hannun Y A. 2006. |
| phosphate | | Biochimica et Biophysica Acta 1761: 927-946. |
| glucosyl-ceramide | HIV gp120 protein | Snook C F, Jones J A, & Hannun Y A. 2006. |
| | Raphanus sativus | Biochimica et Biophysica Acta 1761: 927-946; |
| | Anti-Fungal Protein | Thevissen K, et al. 2004 Defensins from insects and |
| | | plants interact with fungal glucosylceramides. J Biol |
| | | Chem 279: 3900-3905 |
| Cerebroside Sulphate | cardiotoxin | Snook C F, Jones J A, & Hannun Y A. 2006. |
| (Sulfatide) | | Biochimica et Biophysica Acta 1761: 927-946. |
| sphingomyelin | Lysenin | Snook C F, Jones J A, & Hannun Y A. 2006. |
| | | Biochimica et Biophysica Acta 1761: 927-946 |
| ganglioside GM1 | β-amyloid protein | Snook C F, Jones J A, & Hannun Y A. 2006, |
| | cholera toxin | Biochimica et Biophysica Acta 1761: 927-946. |
| | E. coli heat-labile | Sandvig, K. et al. 2010. Protein toxins from plants and |
| | enterotoxin LTI | bacteria: probes for intracellular transport and tools |
| | | in medicine. FEBS Lett 584, 2626-2634. |
| ganglioside GT1 | tetanus toxin | Sandvig, K. et al. 2010. FEBS Lett 584, 2626-2634 |
| ganglioside GD1a | E. coli heat-labile | Sandvig, K. et al. 2010. FEBS Lett 584, 2626-2634 |
| | enterotoxin LTIIb | |
| ganglioside GD1b | botulinum toxin | Sandvig, K. et al. 2010. FEBS Lett 584, 2626-2634 |
| | E. coli heat-labile | |
| | enterotoxin LTlIa | |
| ganglioside Gb3 | Shiga toxin verotoxins | Sandvig, K. et al. 2010. FEBS Lett 584, 2626-2634 |
| phosphorylinositol- | Dahlia merckii Anti- | Thevissen et al 2003, FEMS MicroLetters 226: 169-173 |
| mannosyl-ceramide- | Microbial Protein-1 | |
| phosphoryl-inositol | | |

Lipids which may function as characteristic lipids in the practice of the invention include but are not limited to various proteolipids, glycolipids, sphingolipids, phospholipids, sulfolipids and sterols. For example, such lipids include phosphoinositides such as phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI); various polar lipids such as phosphatidyl-serine (PS), phosphatidyl-glycerol (PG), phosphatidyl-ethanolamine (PE), phosphatidyl-choline (PC), lysophosphatidyl-serine (LPS), lysophosphatidyl-glycerol (LPG), lysophosphatidyl-ethanolamine (LPE), lysophosphatidyl-choline (LPC), phosphatidic acid (PA), lysophosphatidic acid (LPA), sphingosine-1-phosphate (S-1-P), ceramide-1-phosphate (C-1-P), a glycosylphosphatidylinositol (GPI)-protein anchor, glycosylsphingosylinositol (GSI), glycosyl phosphoryl inositol ceramide (GPIC) and sphingomyelin (SM); and various other lipids, including but not limited to galactolipids, glycoceramides, glucosyl-ceramide, galactosceramide, and ergosterol.

In particular, PI-4-P is characteristic of oomycetes; glycosylated ceramide-phosphorylinositol (e.g. Cer-P-Inos-Mannose) and phosphorylinositol-mannosyl-ceramide-phosphoryl-inositol (Cer-P-Inos-Man-P-Inos; M(IP)₂C) are characteristic of fungi; and ceramide-phosphorylinositol (Cer-P-Inos) is characteristic of oomycetes, fungi and trypanosomatids (Olsen, I. and Jantzen, E. (2001) Sphingolipids in Bacteria and Fungi. Anaerobe, 7, 103-112; Takahashi H K et al. 2009. Current relevance of fungal and trypanosomatid glycolipids and sphingolipids: studies defining structures conspicuously absent in mammals. Ann Acad Bras Cienc 81:477-488), ergosterol is specific to fungi and trypanosomatids (Prasad R & Ghannoum M A. 1996. Lipids of Pathogenic Fungi. CRC-Press, Boca Raton, Fla.; Roberts C W, et al. 2003. Fatty acid and sterol metabolism: potential antimicrobial targets in apicomplexan and trypanosomatid parasitic protozoa. Molecular and Biochemical Parasitology 126:129-142).

### General Design of Constructs of the Invention

Constructs with at least one first domain that binds to a characteristic cell surface lipid and at least one second domain that exhibits a desired activity of interest are described herein. Some embodiments contain only one first and one second domain, but this is not always the case. For example, a construct may include one first lipid binding domain but this domain may be attached to two or more other second or effector domains that each possess an activity of interest. Other similar arrangements of domains may be envisioned by those of skill in the art, and all such arrangements are encompassed by the present invention. In the discussion presented herein, the construct is generally referred to a comprising a first and second domain, with the possibility of multi-domain constructs being understood.

In some embodiments, both the first and second domains are proteinaceous in nature (i.e. are comprised of a contiguous chain of amino acids such as a peptide, polypeptide or protein). In this case, the constructs are true fusion or chimeric proteins. In other embodiments, one or both of the domains may not be proteinaceous, or portions of one or both of the domains may not be proteinaceous, in which case the construct may be referred to as a “composite” or chimeric construct (e.g. a two- or multi-domain construct). However, for the sake of simplicity, “first domain” and “second domain” are used to refer to domains of all types, whether proteinaceous or not, and it is understood that discussions related to “fusion proteins” are also generally applicable to constructs which are “composites” (i.e. which contain non-protein elements, segments, portions, etc.).

### First Domain of the Construct

Typically, the first domain of a construct binds to a characteristic lipid with a binding affinity in the range of from about 0.1 nM to about 50 μM, and preferably in the range of from about 5 nM to about 1 μM. The first domains are generally proteinaceous in nature i.e. they are generally comprised of amino acids and may be peptides, polypeptides or proteins, although this need not always be the case. The invention also encompasses other molecules (e.g. small organic molecules, etc.) which specifically or selectively bind to particular lipids. Frequently, if the first domain is proteinaceous, it may include all or an operable (i.e. lipid binding) portion of a naturally occurring lipid binding protein. Those of skill in the art will recognize that many lipid binding proteins and polypeptides may be used in the practice of the invention. In one embodiment, the first domain includes at least one Pleckstrin homology domain (PH domain). A PH domain is a protein domain of approximately 120 amino acids that occurs in a wide range of proteins involved in intracellular signaling or as constituents of the cytoskeleton. Individual PH domains specifically bind to phosphoinositides phosphorylated at different sites within the inositol ring, e.g., some bind phosphatidylinositol (4,5)-bisphosphate but not phosphatidylinositol (3,4,5)-trisphosphate or phosphatidylinositol (3,4)-bisphosphate. Other exemplary first domain constituents include but are not limited to: a protein kinase C domain 1 homology (C1) domain, a protein kinase C domain 2 homology (C2) domain, a Fab 1, YOTB, Vac 1 and EEA1 homology (FYVE) domain, a Phagocytic oxidase homology (PX) domain, an Epsin N terminal Homology (ENTH) domain, a Bin-Amphiphysin-Rvs (BAR) domain, a Four-point-one-protein, Ezrin, Radixin and Moesin homology (FERM) domain, a post synaptic density 95 protein, Drosophila disc large tumor suppressor A, and zonula occludens 1 homology (PDZ) domain, a tubby protein homology (tubby) domain, a defensin, a cathelicidin, a lipid transfer protein. Individual members of said protein families bind with varying specificity to different lipids or sets of lipids (Stahelin R V (2009) Lipid binding domains: more than simple lipid effectors. J Lipid Res 50 Suppl:S299-304).

Those of skill in the art will recognize that such lipid binding proteins may be modified for use in the fusion proteins of the invention. For example, particular lipid binding portions or domains of the protein may be used, and/or mutants or variants of the protein or portions thereof which are adapted for use in the invention by any of several means known to those of skill in the art and for any of a variety of reasons, examples of which include but are not limited to: replacement of amino acids (conservatively or non-conservatively) to create or destroy protease cleavage sites; to improve solubility; to improve or reduce stability; to reduce or increase toxicity; to accommodate changes in the nucleic acid sequence that encodes the protein (e.g. to introduce restriction sites for insertion into a vector); to facilitate isolation or purification (e.g. by adding a histidine or other tag); to increase or decrease binding affinity for a particular lipid; to improve selectivity for a particular lipid; by the addition of targeting or signal sequences or sequences which facilitate uptake of the protein by the cell, as a result of changes to the encoding nucleic acid sequence in order to optimize expression by a particular cell type, etc.

In other embodiments, the first domain is proteinaceous but is comprised of peptides with non-naturally occurring sequences that are identified as capable of selectively or specifically binding a characteristic lipid e.g. via the screening of random peptide libraries.

In yet other embodiments, the first domain is or comprises an antibody or portion thereof (e.g. Fab) that binds to the characteristic lipid, or to a portion of the characteristic lipid. In all cases, binding must be sufficient to allow the activity of interest to be expressed, or to allow uptake of the construct by the cell and hence expression of the activity of interest.

The first domain of the fusion constructs of the invention are capable of specifically or selectively binding to at least one characteristic lipid of interest. Domains that bind “specifically” bind only a lipid with a particular molecular structure (i.e. a lipid with a particular chemical formula and a particular pattern of bonding of atoms in the lipid), or to unique portion of such a lipid molecule. Such domains do not bind to other non-targeted lipids of interest or to portions of other non-targeted lipids of interest. Domains that bind “selectively” exhibit a bias toward binding to the targeted lipid or a portion of a targeted lipid, e.g. in competitive assays, they exhibit an affinity for the targeted lipid which is at least about 10, preferably about 50, more preferably about 100, even more preferably at least about 200, 300, 400, 500, 600, 700, 800, 900 or 1000 fold (or more) greater than their affinity for any other non-targeted lipid. However, those of skill in the art will recognize that, as is the case for “characteristic” lipids, specific or selective domains may be specific or selective relative to a given environment, i.e. if the domain binds to several lipids, but only one of the lipids is present in or is likely to be present in the environment where the fusion protein will be used, then the domain may be considered specific or selective for or in the context of that environment or location or locale.

Examples of protein- or peptide-lipid binding combinations that may be used in the practice of the invention include but are not limited to:

1) pleckstrin-homology (PH) domains of human PEPP1 and FAPP1 proteins are highly specific for PI-3-P and PI-4-P, respectively (Dowler et al., 2000 The Biochemical Journal 351, 19-31).
2) pleckstrin-homology (PH) domain of Arabidopsis Enhanced Disease Resistance-2 (EDR2) NP_{—}001119010.1 GI:186512035 is highly specific for PI-4-P (Vorwerk S, et al. 2007. EDR2 negatively regulates salicylic acid-based defenses and cell death during powdery mildew infections of Arabidopsis thaliana. BMC plant biology 7:35). Arabidopsis EDR2 sequences are presented in FIGS. 8A and B and FIGS. 15 A and B. Exemplary PH domains from soybean and potato ERDs are shown in FIGS. 16A and B and FIGS. 17A and B, respectively.
3) pleckstrin-homology (PH) domain of Arabidopsis phosphatidylinositol-4-kinase (AtPI4K) GenBank AF035936.2 GI:9695358 is highly specific for PI-4-P (Stevenson J M, Perera I Y, & Boss W F. 1998. A phosphatidylinositol 4-kinase pleckstrin homology domain that binds phosphatidylinositol 4-monophosphate. J. Biol. Chem. 273(35):22761-22767) (see FIGS. 18 A and B). Similar sequences from soybean and tobacco are shown in FIGS. 9A and B and FIGS. 20A and B, respectively
4) the peptide Raphanus sativus Anti-Fungal Protein (RsAFP2) GenBank P30230.4 GI:1703206 (see FIG. 9A) and non-toxic mutants thereof (e.g. RsAFP2 mutant Y38G) specifically binds fungal glucosylceramides but not plant or human glucosylceramides (Thevissen K, et al. 2004. Defensins from insects and plants interact with fungal glucosylceramides. J Biol Chem 279:3900-3905).
5) the peptide Dahlia merckii Anti-Microbial Protein-1 (DmAMP1) GenBank P0C8Y4.1 GI:229890071 (see FIG. 9B) binds the fungal-specific glycosphingolipid, phosphorylinositol-mannosyl-ceramide-phosphoryl-inositol (Cer-P-Inos-Man-P-Inos; M(IP)2C) (Thevissen et al 2000, PNAS; 97; 17:9531-9536; Thevissen et al 2003, FEMS MicroLetters 226:169-173).
6) the peptide Bombyx mori cecropin B BAA01889.1 GI:217270 binds the sterol ergosterol that is specific for fungi and trypanosomatid parasites (De Lucca A J, et al. 1998. Fungicidal and binding properties of the natural peptides cecropin B and dermaseptin. Med Mycol 36(5):291-298; Roberts C W, et al. 2003. Fatty acid and sterol metabolism: potential antimicrobial targets in apicomplexan and trypanosomatid parasitic protozoa. Molecular and Biochemical Parasitology 126:129-142. Exemplary silkworm sequences are presented in FIGS. 21A and B.
7) Other exemplary first domain components include but are not limited to: Arabidopsis-PH-domain-protein-1 (AtPH1) PH domain (see FIGS. 22 A and B); and soybean AtPH1-homolog (GmPH1) PH domain (See FIGS. 23 A and B).

### Second Domain of the Construct

Fusion proteins or other compositions of the invention also comprise at least one second domain which possesses an activity of interest with respect to the targeted cell, i.e. the second domain is capable of exerting a desired effect on the cell. In some embodiments (e.g. when the cell is a pathogen or other unwanted cell), the effect may be toxicity to the cell, or inhibition of the cell (e.g. slowing or stopping the cell's metabolism, its ability to reproduce, etc.), or any other desired effect. In other embodiments, for example, when a desirable cell is targeted, the second domain may have an entirely different effect on the cell which may be beneficial to the cell (or to the host organism). For example, the second domain may accelerate growth of or cell division by the cell; or may influence the cell's metabolic capacity; or may cause the cell to produce a product of interest; or may extend the life of the cell. For example, for medicine or agriculture, the second domain may be a nutritional or therapeutic substance that enters a beneficial microbe and stimulates it to increase production of a beneficial substance (e.g. a vitamin, an antibiotic that kills neighboring undesirable microbes, etc.); or the second domain may comprise a therapeutic that enters a microbe and blocks it from producing an undesirable substance, etc. (e.g. many pasture grasses contain endophytic fungi that produce toxins that protect the grass against insects, but are toxic to grazing animals, and the fungi may be targeted according to the methods of the invention); etc. In industry, the second domain may comprise a chemical that enters a targeted microbe in a bioreactor and causes it to commence or increase production of a substance of interest, e.g. a component of biofuel.

In some embodiments, the second domain is proteinaceous in nature and comprises a peptide, polypeptide or protein or portion thereof, which displays or exhibits the activity of interest. If the targeted cells are pathogenic, the second domain typically has a toxic, harmful, damaging or inhibiting effect on the cells. For example, the second domain may be any protein that causes cell death or disruption of growth or metabolism when bound to the plasma membrane of a eukaryotic cell or when internalized into the cytoplasm of a the cell. Preferably, the selectivity of the first binding domain would preclude membrane-binding or entry into other cells, e.g. host plant or animal cells. Examples of such second domain proteins include, but are not limited to, nucleases, proteases, lipases, phosphatases, ATPases, pore-forming peptides, proteins that disrupt the redox balance such as glucose oxidase, or proteins that directly trigger apoptosis such as BAX. The second domain may comprise enzymes which modify the characteristic cell surface lipid, or other proteins, lipids and nucleic acids on or within the cell. Such enzymes include but are not limited to: various hydrolytic enzymes such as phosphatases, phospholipases, etc.; various modifying enzymes such as methylases, acetylases, glycosylases, etc. In other embodiments, the second domain is proteinaceous but has a desired or beneficial non-harmful effect on the cell or the host organism in which the cell is located, as described elsewhere herein.

To further improve the selectivity, and preclude any possibility of toxic effects on host cells, the second domain may also target proteins or other molecules found only in the targeted cells, or else proteins that differ substantially in sequence or structure between the targeted cells and, e.g. a host species in which the cells are located or are likely to infect. Examples include, but are not limited to antibodies (e.g. single chain antibodies, Fab portions of antibodies, etc.) or random peptides that bind to cellular proteins and cause them to be inhibited, degraded or mistargeted. Alternatively, the second domain proteins could be dominant-negative mutants of essential proteins such as protein kinases, transcription factors, ribosomal proteins, cell division proteins, structural proteins, or secretion machinery proteins.

In one embodiment the second domain may consist of short peptides that inhibit essential interactions of specific mitogen-activated-protein kinases (MAP kinases) with other regulatory proteins. MAP kinases regulate large numbers of cellular processes in many eukaryotic organisms. Several MAP kinases are known to be essential for the pathogenicity of fungal and oomycete plant pathogens (Zhao X, Mehrabi R, & Xu J R. 2007. Mitogen-activated protein kinase pathways and fungal pathogenesis. Eukaryotic cell 6:1701-1714; Li A, et al. 2010. PsSAK1, a stress-activated MAP kinase of Phytophthora sojae, is required for zoospore viability and infection of soybean. Mol Plant Microbe Interact 23:1022-1031) (FIGS. 12 A and B). The activity of MAP kinases is regulated by interactions with transcription factors (which they phosphorylate), with MAPK kinase kinases (MKKs) that phosphorylate them, and MAPK kinase phosphatases (MKPs) that dephosphorylate them. Binding of MAP kinases to these regulatory proteins is mediated by a short sequence of amino acids called a docking domain. Both the MAP kinases and their regulatory proteins possess docking domains (Liu S et al. 2006. Structural basis of docking interactions between ERK2 and MAP kinase phosphatase 3. Proc. Natl. Acad. Sci. USA 103:5326-5331; Tanoue T to et al. 2000. A conserved docking motif in MAP kinases common to substrates, activators and regulators. Nat Cell Biol 2:110-116). These docking domains are specific to each protein interaction. When the docking domains are detached from their parent MAP kinase or regulatory protein, they retain the ability to bind their normal target. Thus short peptide sequences having the sequences of the docking domains can inhibit the interactions of MAP kinases with their regulatory domains and so disrupt the functioning of the cell (Fukami Y, et al 1999. Peptide inhibitors of the mitogen-activated protein kinase pathway: a structure-mimetic peptide corresponding to the conserved inter-DFG-APE region in the kinase domain. Pharmacol Ther 82:399-407; Wang X, et al. 2010. Mitogen-activated protein kinase pathway inhibitors: inhibitors for diseases? Front. Med. China 4:46-53). When a MAP kinase inhibitory peptide is connected to a cell entry peptide, it becomes a cell permeable MAP kinase inhibitor (Holzberg D, et al. 2003. Disruption of the c-JUN-JNK complex by a cell-permeable peptide containing the c-JUN delta domain induces apoptosis and affects a distinct set of interleukin-1-induced inflammatory genes. J Biol Chem 278:40213-40223).

### Additional Embodiments of the Construct

In yet other embodiments, multiple first domains may be present in the construct. For example, the construct may comprise a module or domain for entering an infected host cell (e.g. by PI-3-P-binding), a second module for entering a pathogen that is within the host cell (e.g. by PI-4-P-binding); and a third module that can specifically harm or inhibit the function of the pathogen (without harming or inhibiting the host cell).

In some embodiments, the domains of the construct are connected via a link or linking sequence, particularly if both domains are proteinaceous. Exemplary linker of spacer sequences are typically from about 3 to about 12 amino acids in length. They may include proteolytic cleavage sites if it is desirable to release the second domain from the construct, e.g. after uptake by the cell. In other embodiments, the domains may be joined chemically e.g. by covalent bonding between atoms of the first and second domains.

Because characteristic lipids are present on the surface of cells, it is likely that they are essential for the proper functioning of the cell, and that interference with the function may also be a route to preventing or treating infections by pathogens with characteristic surface lipids. In some embodiments, the invention provides active forms of proteins that destroy or interfere with the functioning of characteristic lipids. In a variation of the invention, a single domain agent (e.g. a single protein, polypeptide or peptide) may exhibit both lipid binding activity and an activity of interest that interferes with the function of one or more characteristic lipids (e.g. by sterically blocking the lipid, by chemically modifying the lipid, by cleaving the lipid, etc.).

For instance, for the exemplary oomycete pathogen, PI-4-P presumably serves an important function in the physiology of P. sojae and other oomycetes, either during normal growth or during infection, or both. Without being bound by theory, external PI-4-P may enable the pathogen to measure the external concentration of its effectors by mediating re-entry of certain effectors into the pathogen cytoplasm where they may interact with a receptor. Therefore, proteins that bind to and interfere with the function of PI-4-P on the oomycete membrane could be used for therapeutic treatment of infections or could be secreted by transgenic plants or animals to provide protection against infection. For example, PI-4-P on the oomycete membrane could be sequestered from its normal function by secretion of PI-4-P-binding proteins from plants which are genetically engineered to produce such proteins. Genetic engineering of plants leading to the secretion of enzymes which can bind to and hydrolyze PI-4-P or modify it in other ways may be effective in reducing the level of PI-4-P available for normal function. Examples of such enzymes include but are not limited to PI-4-phosphatases or phospholipases; examples of these enzymes have been described in the literature (Balla, 2007. Imaging and manipulating phosphoinositides in living cells. J Physiol 582:927-937). Additionally, the production, via genetic engineering, of enzymes (e.g. microbial enzymes) that cause modifications of PI-4-P may be utilized, such enzymes including but not limited to methylases, acetylases, glycosylases, etc.

A particularly useful enzyme for use in the genetic engineering of plants and/or plant cells is a phosphotidylinositol-specific phospholipase C that cleaves PI-4-P into 1,4-inositol diphosphate (1,4IP2) and diacylglycerol (Balla, 2007). Not only is the level of PI-4-P reduced as a result of cleavage, but 1,4IP2 is produced simultaneously, and 1,4IP2 is known to inhibit entry of oomycete effectors into plant and animal cells. 1,4IP2 may also inhibit the binding of other proteins to PI-4-P on the oomycete membrane surface that are required for normal PI-4-P function. For example, and without being bound by theory, if re-entry of effector proteins into oomycete hyphae is a normal mechanism for regulating effector biosynthesis, then preventing effector re-entry by both hydrolyzing PI-4-P and by producing 1,4IP2 should effectively disrupt the regulation of effector synthesis, and hence virulence.

### Application or Administration of the Constructs

In some embodiments, the constructs of the invention are produced outside the host cell and the targeted cell is contacted by the construct, e.g. by application of the construct at a location or to an environment where the targeted cell is likely to be. In some embodiments, the constructs are applied to or administered to the host cells or host organisms, particularly when the targeted cell is a pathogen. In other embodiments, the constructs are applied to the habitat of a targeted organism, e.g. to standing water such as swamps; to sources of drinking water, etc. Thus, the invention also provides compositions which contain the constructs and are suitable for such administration or application. The mode of administration will depend on several factors, including the nature of the construct and the host. If the host organism is a plant, application is generally in the form of a foliar spray or watering solution of, e.g., an aqueous or oil solution that includes the construct. For administration to an animal, which may be a human, any suitable composition, many of which are known in the art, may be employed, e.g., various pills, powders, liquids, injectable formulations, etc. Likewise, any suitable means may be used, including but not limited to by injection (e.g. subcutaneous or intramuscular), inhalation, orally, intranasally, by ingestion of a food product containing the construct, etc. In addition, the compositions may include one or more than one construct. For example, a preparation for application to plants may include a construct that binds to characteristic lipids of several different types of pathogens. In addition, the construct may be administered to plants in conjunction with other beneficial substances, such as fertilizers, various pesticides, growth factors, etc. The same is true for administration to animals, where one or more than one type of construct may be administered, and may be administered in conjunction with other beneficial substances such as chemotherapeutic agents that also have activity against a pathogen.

### Genetically Engineered Plant and Animal Cells

Plants, animals or microbes may be genetically engineered so that they produce proteins that contain at least one first domain that binds to a characteristic cell surface lipid and at least one second domain that exhibits a desired activity of interest. Genetically engineered organisms will be protected against pathogens without the need to externally administer a substance. The proteins may be directed inside the engineered cell if it is necessary, for example, to target a pathogenic microbe that invades the interior of the host cell. Alternatively the proteins may be secreted out of the engineered cell if it is necessary, for example, to target a pathogenic microbe that remains outside of the host cells. Alternatively the proteins may be targeted to a specific structure used by the pathogen such as a haustorium (a specialized hypha produced by many fungi and oomycetes that partially invades the interior of a host plant cell).

Those of skill in the art are familiar with methods for the genetic engineering (genetic modification) of plants. This is generally accomplished by introducing genetic material (e.g. one or more genes) encoding the protein of interest into one or more cells of a recipient plant. The nucleic acids may be single or double strand DNA or RNA. Known methods of introducing nucleic acids into plants or plant cells include, for example, microprojectile bombardment, Agrobacterium-mediated techniques, etc. These and other techniques are described, for example, in: U.S. Pat. No. 7,511,205 to Mobel, Jr., (Mar. 31, 2009); U.S. Pat. No. 7,525,028 to Jenkinson (Apr. 28, 2009); U.S. Pat. No. 6,677,507 to de Bruijn (Jan. 13, 2004); and U.S. Pat. No. 6,407,319 to Rose-Pricker et al., (Jun. 18, 2002); and U.S. patent application Ser. No. 10/240,456 (Publication number US 20040053236, McCallum et al., Mar. 18, 2004) the complete contents of each of which is hereby incorporated by reference in entirety.

Those of skill in the art are familiar with techniques for genetically engineering or genetically modifying animal cells, e.g. by the use of vectors such as viral vector (e.g. adenoviral and pox virus vectors), bacterial vectors (e.g. mycobacterial vectors), or by the direct insertion of vectors such as plasmids via e.g. electroporation, by the use of skin or membrane permeating agents, etc. The invention also encompasses nucleic acid sequences and vectors which encode the constructs of the invention.

Those of skill in the art are familiar with techniques for genetically engineering or genetically modifying microbial cells, e.g. for example, protoplast fusion methods, microprojectile bombardment, Agrobacterium-mediated techniques, electroporation methods, etc etc. The invention also encompasses nucleic acid sequences and vectors which encode the constructs of the invention.

Pathogens and Other Symbionts that May be Targeted

Many types of invasive pathogens may be targeted by the methods of the invention. Examples of such pathogens include but are not limited to: any Phytophthora species, e.g. Phytophthora infestans, Phytophthora sojae, Phytophthora ramorum, Phytophthora parasitica, Phytophthora capsici, Phytophthora nicotianae, Phytophthora Phytophthora cryptogea, Phytophthora drechsleri, Phytophthora cactorum, Phytophthora cambivora, Phytophthora citrophthora, Phytophthora citricola, Phytophthora megasperma, Phytophthora palimivora, Phytophthora megakarya, Phytophthora boehmeriae, Phytophthora kernoviae, Phytophthora erythroseptica, Phytophthora fragariae, Phytophthora heveae, Phytophthora lateralis, Phytophthora syringae; any Pythium species, e.g. Pythium ultimum, Pythium aphanidermatum, Pythium irregulars, Pythium graminicola, Pythium arrhenomanes, Pythium insidiosum; any downy mildew species; any Peronospora species, e.g. Peronospora tabacina, Peronospora destructor, Peronospora sparse, Peronospora viciae; any Bremia species, e.g. Brenda lactucae; any Plasmopora species, e.g. Plasmopora viticola, Plasmopora halstedii; any Pseudoperonospora species, e.g. Pseudoperonospora cubensis, Pseudoperonospora humuli; any Sclerospora species e.g. Sclerospora graminicola; any Peronosclerospora species, e.g. Peronosclerospora pliilippirresis, Peronosclerospora sorghi, Peronosclerospora sacchari; any Sclerophthora species, e.g. Sclerophthora rayssiae, Sclerophthora macrospora; any Albugo species, e.g. Albugo candida; any Aphanomyces species, e.g. Aphanomyces cochlioides, Aphanomyces euteiches, Aphanomyces invadans; any Saprolegnia species, e.g. Saprolegnia parasitica; any Achlya species; any rust fungi; any smut fungi; any bunt fungi; any powdery mildew fungi; any Puccinia species, Puccinia striiformis, Puccinia graminis, Puccinia triticina (syn. Puccinia recondita), Puccinia sorghi, Puccinia schedonnardii, Puccinia cacabata; any Phakopsora species, e.g. Phakopsora pachyrhizi, Phakopsora gossypii; any Phoma species, e.g. Phoma glycinicola; any Ascochyta species, e.g. Ascochyta gossypii; any Cryphonectria species, e.g. Cryphonectria parasitica; any Magnaporthe species, e.g. Magnaporthe oryzae; any Gaeumannomyces species, e.g. Gaeumannomyces graminis; any Synchytrium species, e.g. Synchytrium endobioticum; any Ustilago species, e.g. Ustilago maydis, Ustilago tritici, Ustilaginoidea virens; any Tilletia species, e.g. Tilletia indica, Tilletia caries, Tilletia foetida, Tilletia barclayana; any Erysiphe species, e.g. Erysiphe necator (formerly Uncinula necator); any Blumeria species, e.g. Blumeria graminis; Podosphaera oxyacanthae; any Alternaria species, e.g. Alternaria alternata; any Botrytis species, e.g. Botrytis cinerea; any Diaporthe species, e.g. Diaporthe phaseolorum; any Fusarium species, e.g. Fusarium graminearum, Fusarium oxysporum (e.g. f.sp. lycopersici), Fusarium moniliforme, Fusarium solani; any Leptosphaeria species, e.g. Leptosphaeria macularis, Leptosphaeria maydis; any Macrophomina species, e.g. Macrophomina phaseolina; any Monilinia species, e.g. Monilinia fructicola; any Mycosphaerella species, e.g. Mycosphaerella graminicola, Mycosphaerella fijiensis, Mycosphaerella tassiana, Mycosphaerella zeae-maydis; any Phialophora species, e.g. Phialophora gregata; any Phymatotrichopsis species, e.g. Phymatotrichopsis omnivora; any Taphrina species, e.g. Taphrina deformans; any Aspergillus species, e.g. Aspergillus flavus, Aspergillus parasiticus, Aspergillus fionigatus; any Verticillium species, e.g. Verticillium dahliae, Verticillium albo-atrum, Rhizoctonia solani, Ophiostoma ulmi (syn. Ceratocystis ulmi), Ophiostoina novo-ulmi; any Septoria species, e.g. Septoria avenae; any Pyrenophora species, e.g. Pyrenophora tritici-repentis; any Colletotrichum species, e.g. Colletotrichum graminicola; any Sclerotinia species, e.g. Sclerotinia sclerotiorum; any Sclerotium species, e.g. Sclerotium rolfsii; any Thielaviopsis species, e.g. Thielaviopsis basicola; any Coccidioides species, e.g. Coccidioicles immitus; any Paracoccidioides species, e.g. Paracoccidioides braziliensis; any Pneumocystis species, e.g. Pnezonocystis carinii; any Histoplasina species, e.g. Histoplasma capsulatum; any Cryptococcus species, e.g. Cryptococcus neoformans; any Candida species, e.g. Candida albicans; any apicomplexan parasite species such as: any Plasmodium species, e.g. Plasmodium falciparum, Plasmodium vivax, Plasmodium ovale, Plasmodium malariae; any Babesia species, e.g. Babesia bovis, Babesia bigemina; any Cryptosporidium species, e.g. Cryptosporidium parvum; any Toxoplasma species, e.g. Toxoplasma gondii; any Trypanosomatid species such as: any Trypanosoma species, e.g. Trypanosoma brucei, Trypanosoma cruzi, Trypanosoma congolense, Trypanosoma vivax; any Leishmania species, e.g. Leismania donovani; any amebozoan parasites; any Entamoeba species, e.g. Entamoeba histolytica; any Mastiganzoeba species; any Schistosoma species; any Onchocerca species; any Brugia malayi species; any Meloidogyne species; any Heterodera species; any Giardia species; any microsporidial species; any Enterocytozoon species; any Encephalitozoon species, e.g. Encephalitozoon cuniculi; any parasite; any parasitic plant; any parasitic alga; any myco-heterotrophic plant; any Triphysaria species; any Striga species; any Cuscuta species; any parasitic animal; any bacterial or archaebacterial species; any pathogenic bacterial or archaebacterial species; any symbiotic microbe; any symbiotic bacterium; any symbiotic archaebacterium; any symbiotic fungus, any symbiotic oomycete; any symbiotic protozoan; any symbiotic nematode; any symbiotic trematode; any symbiotic alga; any symbiotic animal; any symbiotic plant; any endophytic microbe; any endosymbiotic microbe; any endosymbiotic bacterium; any endosymbiotic archaebacterium; any endosymbiotic fungus, any endosymbiotic oomycete; any endosymbiotic protozoan; any endosymbiotic nematode; any endosymbiotic trematode; any endosymbiotic bacterium; any endosymbiotic archaebacterium; any endosymbiotic fungus, any endosymbiotic oomycete; any endosymbiotic protozoan; any endosymbiotic nematode; any endosymbiotic trematode; any endosymbiotic alga; any endosymbiotic animal; any endosymbiotic plant; any episymbiotic microbe; any episymbiotic bacterium; any episymbiotic archaebacterium; any episymbiotic fungus, any episymbiotic oomycete; any episymbiotic protozoan; any episymbiotic nematode; any episymbiotic trematode; any episymbiotic bacterium; any episymbiotic archaebacterium; any episymbiotic fungus, any episymbiotic oomycete; any episymbiotic protozoan; any episymbiotic nematode; any episymbiotic trematode; any episymbiotic alga; any episymbiotic animal; any episymbiotic plant; any endophytic bacterium; any endophytic archaebacterium; any endophytic fungus, any endophytic oomycete; any endophytic protozoan; any endophytic nematode; any endophytic trematode; any endophytic bacterium; any endophytic archaebacterium; any endophytic fungus, any endophytic oomycete; any endophytic protozoan; any endophytic nematode; any endophytic trematode; any endophytic alga; any endophytic animal; any endophytic plant; any epiphytic microbe; any epiphytic bacterium; any epiphytic archaebacterium; any epiphytic fungus, any epiphytic oomycete; any epiphytic protozoan; any epiphytic nematode; any epiphytic trematode; any epiphytic bacterium; any epiphytic archaebacterium; any epiphytic fungus, any epiphytic oomycete; any epiphytic protozoan; any epiphytic nematode; any epiphytic trematode; any epiphytic alga; any epiphytic animal; any epiphytic plant; any rhizosphere microbe; any rhizosphere bacterium; any rhizosphere archaebacterium; any rhizosphere fungus, any rhizosphere oomycete; any rhizosphere protozoan; any rhizosphere nematode; any rhizosphere trematode; any rhizosphere bacterium; any rhizosphere archaebacterium; any rhizosphere fungus, any rhizosphere oomycete; any rhizosphere protozoan; any rhizosphere nematode; any rhizosphere trematode; any rhizosphere alga; any rhizosphere animal; any rhizosphere plant; any mycorrhizal fungus; any ectomycorrhizal fungus; any endomycorrhizal fungus; any arbuscular mycorrhizal fungus; any endo-ecto-mycorrhizal fungus; any ericoid mycorrhizal fungus; any Glomus species; any Gigaspora species; any Acaulospora species; any Tuber species; any Trichoderma species; any Epichloe species; any Neotyphodiun species; any Taxomyces species; any Nodulisporium species etc.

In some embodiments, the targeted cell is not the pathogen per se but is a cell infected by a pathogen which, as a result of the infection or for other reasons, produces a characteristic lipid on its surface. In one embodiment, the presence of the intracellular pathogen results in the appearance of a new, specific lipid on the infected cell. In this case, the construct penetrates the infected cell via the lipid binding domain, and the second domain (or a plurality of second domains) has/have the ability to (i) kill the pathogen; and/or (ii) stimulate the infected cell to kill the pathogen, and/or (iii) neutralize molecules produced by the pathogen to prevent such killing; and/or (iii) kill the host cell outright, thus preventing maturation or replication of the contained pathogen.

In further embodiments, the targeted cells are unwanted cells which display unregulated or uncontrolled growth, such as cancer cells or non-cancerous growths. In other embodiments, the targeted cells are pathological cells, meaning any unwanted or malfunctioning cells which are identified as displaying characteristic lipids, examples of which include but are not limited to: adipose tissue cells that are no longer correctly responding to insulin; neurons that are not correctly releasing, re-outpacing or responding to neurotransmitters such as dopamine; thyroid cells that are under-producing or over-producing thyroxine; hypothalamus cells that are under-producing or over-producing a certain hypothalamic-releasing hormone; pituitary cells that are under-producing or over-producing a pituitary hormone; adrenal gland cells that are under-producing or over-producing an adrenal hormone, etc. According to the invention, such cells may be destroyed by the methods described herein. In other embodiments, such cells may be treated by the delivery, to the cells, of a therapeutic substance that improves or ameliorates the functioning of the cell. For example, in some cases, transcription factors delivered via a lipid-binding protein may be an effective therapeutic to correct the cells' malfunction.

In further embodiments, the targeted cells are pathological cells such as a host cell that has become infected with a type of pathogen that requires the host cell to remain alive in order to persist, reproduce, proliferate or spread, examples of which pathogens include but are not limited to: a virus, an archaebacterium, a bacterium, a fungus, an oomycete, an apicomplexan parasite, a trypanosomatid parasite, an amoebozoan parasite, a nematode parasite, a trematode parasite, a microsporidial parasite, an algal parasite, a plant parasite, an animal parasite, downy mildew, Bremia, Hyaloperonospora, Peronospora, Sclerospora, Peronosclerospora, Sclerophthora, Albugo, Puccinia, Phakopsora, Magnaporthe, Gaeumannomyces, Synchytrium, Ustilago, Tilletia, Erysiphe, Blumeria, Fusarium, Leptosphaeria, Coccidioides, Paracoccidioides, Pneumocystis, Histoplasma, Cryptococcus, Plasmodium, Babesia, Cryptosporidium, Toxoplasma, Trypanosoma, Leishmania, Giardia, Enterocytozoon, and Encephalitozoon, Triphysaria, Striga, Cuscuta. Human Immunodeficiency Virus, influenza virus, Epstein-Barr Virus, varicella-zoster (chicken pox) virus, hepatitis B virus, adenovirus, any pox virus, variola major (smallpox) virus, any hemorrhagic fever virus, Ebola virus, Marburg virus, Lassa fever virus, Crimean-Congo hemorrhagic fever virus any arenavirus, lymphocytic choriomeningitis arenavirus, Junin virus, Machupo virus, guanarito virus, any bunyavirus, rift valley fever bunyavirus, any hantavirus, any flavivirus, dengue virus, any filovinis, any calicivirus, hepatitis A virus, any encephalitis virus, west nile virus, lacrosse virus, California encephalitis virus, Venezuelan equine encephalitis virus, eastern equine encephalitis virus, western equine encephalitis virus, Japanese encephalitis virus, Kyasanur forest virus, yellow fever virus, rabies virus, Chikungunya virus, severe acute respiratory syndrome-associated (SARS) coronavirus, Francisella, Burkholderia, Coxiella, Brucella, Chlamydia, Mycobacterium, any Rickettsia, Rickettsia prowazekii (Typhus fever), Listeria, Cyclospora, and Entamoeba.

Plants and Animals that may Benefit from the Practice of the Invention

Examples of plants and/or plant cells that can benefit from the practice of the invention include but are not limited to: wheat, maize, rice, sorghum, barley, oats, millet, soybean, common bean (e.g. Phaseolus species), green pea (Pisum species), cowpea, chickpea, alfalfa, clover, tomato, potato, tobacco, pepper, egg plant, grape, strawberry, raspberry, cranberry, blueberry, blackberry, hops, walnut, apple, peach, plum, pistachio, apricot, almond, pear, avocado, cacao, coffee, tea, pineapple, passion fruit, coconut, date and oil palm, citrus, safflower, carrot, sesame, common bean, banana, citrus (e.g. orange, lemon, grapefruit), papaya, macadamia, guava, pomegranate, pecan, Brassica species (canola, cabbage, cauliflower, mustard etc), cucurbits (pumpkin, cantaloupe, squash, zucchini, melons etc), cotton, sugar cane, sugar beets, sunflower, lettuce, onion, garlic, ornamental cut flowers, grasses used in lawns, athletic fields, golf courses and pastures (e.g. Festuca, Lolium, Zoysia, Agrostis, Cynodon, Dactylis, Phleum, Phalaris, Poa, Bromua and Agropyron species); trees such as oak, chestnut (e.g. American chestnut) etc.

Examples of animals and/or animal cells that may benefit from the practice of the invention include but are not limited to: various mammals such as humans, cattle, sheep, pigs, goats, horses, donkeys, cats, dogs, rabbits, llamas, buffalo, bison, mink, chinchilla, etc.; chickens; turkeys; emus; ostriches; bees; fish such as salmon, trout, bass, catfish, etc.; shellfish such as crayfish, lobsters, shrimp, crabs, clams, mussels, etc.

In another embodiment, plants such as grasses could be engineered to produce a protein that enters the fungi and blocks toxin production, by, for example, binding to the promoters of the toxin biosynthesis genes).

### Exemplary Embodiment

### Oomycetes

In an exemplary embodiment, the invention provides compositions and methods for the prevention and treatment of diseases caused by oomycetes. Oomycetes are filamentous eukaryotic organisms, which, in their mature form, contain multiple coenocytic (non-septate) hyphae. The discovery that PI-4-P is present on the hyphae of oomycetes but not on the surface of plant or animal cells permits selective targeting of oomycetes via PI-4-P in order to prevent or treat diseases and disorders they cause. For example, in one embodiment of the invention, this discovery has led to the development of fusion proteins in which a PI-4-P-binding domain is fused to a protein or polypeptide that is toxic or inhibitory to oomycetes. When oomycetes are exposed to the fusion proteins, the fusion proteins selectively enter oomycete hyphae, but not plant or animal cells, and are toxic to the oomycete. Such proteins, discussed in detail below, may be used for the therapeutic prevention and/or treatment oomycete infections.

Phospholipids such as PI-4-P thus act as a gateway for the entry into a cell of interest of at least one agent of choice, e.g. an agent that kills, damages or inhibits the oomycete and thus prevents or treats diseases caused by oomycetes. Experiments conducted with the exemplary oomycete pathogen Phytophthora sojae have demonstrated that PI-4-P-binding proteins enter P. sojae hyphae via binding to the surface PI-4-P. This finding has led to the design of proteins which contain at least one PI-4-P-binding domain and at least one domain that is toxic to oomycetes. Such chimeric (composite, fusion) proteins bind to and enter pathogenic oomycetes via PI-4-P on the outer surface of hyphae, and, once the protein is internalized, the toxic portion of the molecule kills or damages the oomycete. Significantly, such proteins cannot enter plant or animal cells which do not contain PI-4-P on their cell surfaces, rendering them immune to protein entry and the effects of the toxin.

This finding is in contrast to the occurrence of phosphatidylinositol-3-phosphate (PI-3-P) on the outer surface of the plasma membrane of plant cells and some animal cells which has been previously described (U.S. patent application Ser. No. 12/468,470 filed May 19, 2009, published as US 2010-0093601; and U.S. patent application Ser. No. 12/944,345 filed Nov. 11, 2010, published as U.S. Pat. No. ______; the complete contents of both of which are hereby incorporated by reference). Proteins that bind PI-3-P, including oomycete and fungal pathogen effector proteins, can enter plant cells and some animal cells via binding the surface PI-3-P, and moieties and methods to block this binding are described in the referenced applications.

The invention is further illustrated by the following examples, which should not be construed as limiting in any way.

### EXAMPLES

### Example 1

### PI-4-P-Binding Proteins Can Enter Hyphae of the Oomycete P. sojae

To test for the presence of PI-3-P and PI-4-P on P. sojae hyphae, and the ability of those phosphoinositides to carry binding proteins into the hyphae, the pleckstrin-homology (PH) domains of the human proteins phosphatidylinositol-3-phosphate-binding PH-domain protein-1 (PEPP1) and phosphatidylinositol-4-phosphate adaptor protein-1 (FAPP1), respectively, were utilized (Dowler et al., 2000). (The sequence of full length naturally occurring FAPP1 is shown in FIGS. 4 A and B, and the sequence of synthetic FAPP1 including attB sites used for Gateway homologous recombination cloning are shown in FIGS. 5A and B.) PH domains in general mediate phosphoinositide binding, and the PH domains of PEPP1 and FAPP1 are highly specific for PI-3-P and PI-4-P, respectively (Dowler et al., 2000). To create biosensors capable of detecting PI-3P and PI-4-P in vivo, the PH domain of PEPP1 was fused to green fluorescent protein (GFP) and the PH domain of FAPP1 was fused to the modified red fluorescent protein, mCherry. (Sequences of the nucleic acids used for the production of these proteins, and the amino acid sequences corresponding to the same, are presented in FIGS. 6 A and B (GST-FAPP1-GFP) and FIGS. 7 A and B (GST-FAPP1-mCherry. PEPP1-GFP and FAPP1-mCherry proteins (1 mg/ml in 25 mM 2-(N-morpholino)ethanesulfonic acid (MES) pH 5.8) were incubated with P. sojae hyphae for 6 hr at 25° C. then washed for 30 min with 25 mM MES pH 5.8, before being photographed using a Zeiss LSM510 laser scanning confocal microscope with an argon laser excitation wavelength of 488 nm for GFP or with a HeNe laser at a wavelength of 543 nm for mCherry.

FIG. 1A shows that FAPP1-mCherry bound to the membrane of P. sojae hyphae and also entered into the cells in abundance. FIG. 1B shows hyphae stained simultaneously with PEPP1-GFP and FAPP1-mCherry. These pictures show strong membrane binding and cellular entry by FAPP1-mCherry but not by PEPP1-GFP. In contrast, when plant or human cells are stained with PEPP1-GFP and FAPP1-mCherry, there is strong membrane binding and cellular entry by PEPP1-GFP but not by FAPP1-mCherry (FIG. 1C, D).

These results demonstrate that PI-4-P occurs on the outer membrane surface of P. sojae hyphae, and that binding of a protein to PI-4-P is sufficient for a substantial amount of that protein to enter into the cytoplasm of the hyphae. In contrast, PI-4-P-binding proteins do not enter plant or human cells.

The results shown in FIG. 1 also demonstrate how to verify that a lipid-binding module (a PI-4-P or PI-3-P-binding module in this example) has the ability to carry a desired cargo domain inside the targeted cell. By using a fluorescent protein (GFP or mCherry in the example shown in FIG. 1) as the cargo domain, confocal microscopy can be used to observe directly the location of the protein. For example, FIG. 1D shows that FAPP1 is capable of carrying a cargo (mCherry) into oomycete cells but PEPP1 is not capable of carrying a cargo (GFP) into the same cells.

### Example 2

### Phospholipid-Binding Specificity of PEPP1 and FAPP1 Biosensors

PEPP1-PH and FAPP1-PH domains were tested for phospholipid binding as fusions with GFP at the C-terminus. Lipid filters were prepared by spotting 1 μl of each lipid at an appropriate series of dilutions onto Hybond-C-extra membranes (GE Healthcare). After blocking of the filter, the respective fusion protein (20 μg) was added and incubated overnight at 4° C. After washing, bound proteins were detected with rabbit anti-GFP antibody followed by peroxidase-conjugated anti-rabbit antibody and ECL reagent.

The results presented in FIG. 2 provide an example of how to validate the lipid-binding specificity of a protein or protein domain intended to be used for specific binding to a characteristic lipid.

### Example 3

### Genetic Engineering of Plant Cells so that they Secrete PI-4-P Binding Proteins

In order to deliver fusion proteins that can enter and inhibit, damage or kill pathogens that are infecting plant tissues, it is convenient to genetically engineer the plants so that they secrete the proteins, either constitutively or at elevated levels (10-fold, 100-fold 1000-fold or more) during infection. This approach avoids the need to spray the plants or coat plant seeds with inhibitory proteins or other compounds. A commonly used method of creating genetically engineered plants is to use Agrobacterium tumefaciens cells to deliver the DNA sequences of interest into the plant cells. We have created DNA sequences that encode a fusion protein consisting of the signal peptide of the secreted soybean protein, PR1a, a FAPP1 PH domain that binds PI-4-P and green fluorescent protein (GFP) (see FIGS. 13 A and B). The purpose of the PR1a signal peptide is target the FAPP1-GFP fusion to be secreted out of the N. benthamiana cells. We have used Agrobacterium cells to deliver the DNA sequences into cells of the plant Nicotiana benthamiana and have validated that the transformed cells secrete abundant amounts of the fusion protein (FIG. 3). The fusion protein can be observed to accumulate in the apoplast (marked “a” in FIG. 3). The apoplastic location of the protein can most clearly be observed when the plant cells are plasmolysed (FIG. 3B). Fusion protein that is still within the vesicles (“v”) of the secretory system can also be observed. These results validate that a PI-4-P-binding protein such as FAPP1 can be efficiently secreted from a plant cell and can accumulate abundantly in the apoplast. The same procedure may be used to validate the secretion and accumulation of any protein that binds a characteristic lipid, from any plant cell that can be transformed using Agrobacterium cells.

### Example 4

### Inhibition of Candida albicans Cells with a Dominant-Negative YPT1 Protein that Selectively Enters Yeast Cells

Candida albicans is a fungus that is a common resident of skin and mucosal surfaces of humans and other animals. Under some conditions it can proliferate extensively and cause disease of mucosal tissues. Occasionally it can also enter the blood stream where it can cause a lethal systemic infection. C. albicans is closely related to the model fungus, Saccharomyces cerevisiae. C. albicans secretes many proteins, such as proteases, as part of its machinery for causing infection in humans and other mammals. One protein that is an essential component of the secretory apparatus of C. albicans is the protein YPT1 (the nucleic acid, SEQ ID NO: 13 and encoded amino acid sequence, SEQ ID NO: 14, each of which are shown in FIGS. 10 A and B, respectively. A dominant-negative mutant of YPT1, ypt1(N121I), (see FIGS. 11A and B, SEQ ID NOS: 15 and 16) can interact with the other proteins of the secretory apparatus, but cannot execute its normal function, therefore disrupting the entire apparatus, and inhibiting growth, secretion and virulence (Lee S A et al. 2001. Overexpression of a dominant-negative allele of YPT1 inhibits growth and aspartyl protease secretion in Candida albicans. Microbiology 147:1961-1970). However, ypt1(N121I) protein cannot enter C. albicans (or any other) cells and so cannot be used as therapeutic by itself. C. albicans cells carry on their membrane surface the characteristic lipid glucosyl-ceramide, which renders them sensitive to the defensin RsAFP2, which binds specifically to fungal glucosyl-ceramide (Thevissen K, et al. 2004. Defensins from insects and plants interact with fungal glucosylceramides. J Biol Chem 279:3900-3905) A mutant form of RsAFP2 (Y38G) binds glucosylceramide without killing C. albicans cells. A fusion protein that contains RsAFP2-Y38G as its first domain and ypt1(N121I) as its second domain is designed and produced. The fusion protein will enter and inhibit C. albicans cells.

C. albicans cells also carry a second characteristic lipid on their surface, namely phosphorylinositol-mannosyl-ceramide-phosphoryl-inositol (M(IP)2C) (Wells G B, Dickson R C, & Lester R L. 1996. Isolation and composition of inositolphosphorylceramide-type sphingolipids of hyphal forms of Candida albicans. J Bacteriol 178:6223-6226). Dahlia merckii Anti-Microbial Protein-1 (DmAMP1) is a peptide that binds cell surface M(IP)2C. A fusion of DmAMP1 to ypt1(N121I) is designed and produced. The fusion protein will enter and kill C. albicans cells. The RsAFP2-ypt1(N121I) and DmAMP1-ypt1(N121I) proteins can be readily produced in a bacterial expression systems such as E. coli, using standard methods, as neither domain is toxic to bacteria. The proteins, synthesized in and purified from the bacteria are then used as a topical therapeutic for mucosal C. albicans infections or delivered intravenously to treat C. albicans infections. Topical and IV administration result in killing of C. albicans cells and amelioration of the symptoms of infection.

### Example 5

### Genetic Engineering of Soybean to Secrete a Peptide that Enters Phytophthora sojae Hyphae and Inhibits the Essential MAP Kinase PsSAK1

Mitogen-activated protein kinase (MAPK) pathways are universal and evolutionarily conserved signal transduction modules in all eukaryotic cells. PsSAK1 encodes a stress-activated MAPK of Phytophthora sojae (Li A, et al. 2010. PsSAK1, a stress-activated MAP kinase of Phytophthora sojae, is required for zoospore viability and infection of soybean. Mol Plant Microbe Interact 23:1022-1031). PsSAK1 is highly conserved in oomycetes. Reverse-transcription polymerase chain reaction analysis showed that PsSAK1 expression was up-regulated in zoospores and cysts and during early infection (Li A, et al. 2010. Mol Plant Microbe Interact 23:1022-1031). In addition, its expression was induced by osmotic and oxidative stress mediated by NaCl and H₂O₂, respectively. To elucidate the function, the expression of PsSAK1 was silenced using stable transformation of P. sojae. The silencing of PsSAK1 did not impair hyphal growth, sporulation, or oospore production but severely hindered zoospore development, in that the silenced strains showed quicker encystment and a lower germination ratio than the wild type (Li A, et al. 2010. Mol Plant Microbe Interact 23:1022-1031). PsSAK1-silenced mutants produced much longer germ tubes and could not colonize either wounded or unwounded soybean leaves (Li A, et al. 2010. Mol Plant Microbe Interact 23:1022-1031). Thus PsSAK1 is an important regulator of zoospore development and pathogenicity in P. sojae.

Signaling efficiency and specificity of MAP kinases are modulated in large part by docking interactions between individual MAP kinase and the kinase interaction motif (KIM), in its interacting kinases, phosphatases, scaffolding proteins, and substrates (Liu 5, et al. 2006. Structural basis of docking interactions between ERK2 and MAP kinase phosphatase 3. Proc. Natl. Acad. Sci. USA 103:5326-5331). Each MAP kinase carries a KIM docking site located opposite the active site of the kinase (Liu S, et al. 2006. Proc. Natl. Acad. Sci. USA 103:5326-5331). The KIM docking site of PsSAK1 is located between amino acids 296 and 539. Therefore a truncated fragment of PsSAK1 that spans from amino acids 296 to 539 will compete with PsSAK1 for binding to its normal substrates that are important for enabling zoospore development and pathogenicity, and will therefore inhibit zoospore development and pathogenicity when present in the cytoplasm of P. sojae hyphae. PsSAK1(296-539) cannot however enter P. sojae hyphae externally. On the other hand, the FAPP1-PH domain can bind PI-4-P and can carry proteins fused to it into P. sojae hyphae. Therefore a fusion protein consisting of FAPP1-PH as its first domain and PsSAK1(296-539) as its second domain will enter P. sojae and inhibit zoospore development and pathogenicity, by interfering with the normal function of PsSAK1. The host plant infected by P. sojae is soybean. In order to protect soybean against P. sojae infection, transgenic soybean plants are constructed that contain DNA sequences encoding a fusion protein with three modules. The first module consists of a signal peptide, derived from the secreted soybean protein PR1a, the second module is FAPP1-PH, and the third module is PsSAK1(296-539).

Transgenic soybean plants are constructed by using particle bombardment of soybean embryogenic suspension cells (Finer J J & McMullen M D. 1991. Transformation of soybean via particle bombardment of embryogenic suspension culture tissue. In Vitro Cellular & Developmental Biology—Plant 27:175-182). Each transgenic line is checked for the secretion of the FAPP1-PH-PsSAK1(269-539) by using an anti-FAPP1 antibody. Those transgenic plants with high levels of expression are evaluated for P. sojae resistance using well-established greenhouse and growth chamber assays that predict field resistance very well (Olah, A. F. and Schmitthenner, A. F. 1985. A growth chamber test for measuring Phytophthora root rot tolerance in soybean [Glycine max] seedlings. Phytopathology. 75(5): 546-548; Thomison, P. R., Thomas, C. A., and Kenworthy, W. J. (1991) Tolerant and root resistant soybean cultivars: Reactions to Phytophthora rot in inoculum-layer tests. Crop Sci. 31: 73-75). Transgenic plant with high levels of expression are partially or fully resistant to P. sojae.

### Example 6

### Genetic Engineering of Salmon to Secrete a Peptide that Enters Saprolegnia Parasitica Hyphae and Inhibits the Essential Map Kinase SpSAK1

Pathogenic oomycetes of the genus Saprolegnia (order Saprolegniales) cause Saprolegniosis, a disease that is characterized by visible white or grey patches of filamentous mycelium on the body or fins of freshwater fish. Saprolegnia parasitica is economically one of the most important fish pathogens, especially on catfish, trout and salmon species, such as the Atlantic salmon Sahno solar. The high density of fish in aquaculture farms has exacerbated disease problems. S. parasitica causes millions of dollar losses to the aquaculture business worldwide.

S. parasitica has a MAP kinase gene that encodes a protein nearly identical to PsSAK1 (399 of 580 amino acid residues are identical). The KIM docking site of SpSAK1 is located between amino acids 303 and 544. Therefore a truncated fragment of SpSAK1 that spans from amino acids 303 to 544 will compete with SpSAK1 for binding to its normal substrates that are important for enabling zoospore development and pathogenicity, and will therefore inhibit zoospore development and pathogenicity when present in the cytoplasm of S. parasitica hyphae. Since SpSAK1 has no sequences that enable entry into fish cells, a binding domain for a characteristic lipid such as phosphatidylinositol-4-phosphate (FAPP1-PH) is fused to the SpSAK1(275-544) protein, together with a signal peptide that directs secretion of the protein from fish skin cells so that the protein accumulates in the slime layer that coats the fish. An exemplary construct of this type is shown in FIGS. 14 A and B.

DNA sequences encoding the three-module fusion protein are introduced into the ooplasm of fertilized salmon eggs by microinjection (Chourrout D, Guyomard R, & Houdebine L-M. 1986. High efficiency gene transfer in rainbow trout (Salmo gairdneri Rich.) by microinjection into egg cytoplasm. Aquaculture 51:143-150). The microinjected eggs are allowed to develop, and normal fish that develop are tested for the presence and expression of the transgene in the germline (sperm or eggs). Offspring deriving from transgenic sperm or eggs are tested for resistance to Saprolegnia parasitica using an in vivo assay (Stueland, S., Hatai, K. and Skaar, I. 2005. Morphological and physiological characteristics of Saprolegnia spp. strains pathogenic to Atlantic salmon, Salmo salar L. J. Fish Diseases, 28, 445-453), and those which express the transgene are partially or fully resistant to infection by Saprolegnia parasitica.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A fusion construct comprising
at least one first domain specific or selective for binding to a characteristic lipid on the surface of a cell; and
at least one second domain with an activity of interest.

2. The fusion construct of claim 1, wherein said cell is a pathogen or other symbiont.

3. The fusion construct of claim 1, wherein said cell is a cancer cell or other pathological cell displaying a characteristic lipid.

4. The fusion construct of claim 3, wherein said pathological cell is a host cell infected by a pathogen.

5. The fusion construct of claim 2, wherein said pathogen or other symbiont is of a type selected from the group consisting of: an archaebacterium, a bacterium, a fungus, an oomycete, an apicomplexan parasite, a trypanosomatid parasite, an amoebozoan parasite, a nematode parasite, a trematode parasite, a microsporidial parasite, an algal parasite, a plant parasite, an animal parasite, Phytophthora, Pythium, downy mildew, Bremia, Hyaloperonospora, Peronospora, Sclerospora, Peronosclerospora, Sclerophthora, Albugo, Aphanomyces, Saprolegnia, Achlya, Puccinia, Phakopsora, Phoma, Ascochyta, Cryphonectria, Magnaporthe, Gaeumannomyces, Synchytrium, Ustilago, Tilletia, Erysiphe, Blumeria, Alternaria, Botrytis, Diaporthe, Fusarium, Leptosphaeria, Macrophomina, Monilinia, Mycosphaerella, Phialophora, Phymatotrichopsis, Taphrina, Aspergillus, Verticillium, Septoria, Pyrenophora, Colletotrichum, Sclerotinia, Sclerotium, Thielaviopsis, Coccidioides, Paracoccidioides, Pneumocystis, Histoplasma, Cryptococcus, Candida, Plasmodium, Babesia, Cryptosporidium, Toxoplasma, Trypanosoma, Leishmania, Entamoeba, Mastigamoeba, Schistosoma, Onchocerca, Giardia, Enterocytozoon, and Encephalitozoon, Glomus, Gigaspora, Acaulospora, Tuber, Trichoderma, Epichloe, Neotyphodium, Taxomyces, Nodulisporium, Triphysaria, Striga, and Cuscuta.

6. The fusion construct of claim 4, wherein said pathogen is of a type selected from the groups consisting of a virus, an archaebacterium, a bacterium, a fungus, an oomycete, an apicomplexan parasite, a trypanosomatid parasite, an amoebozoan parasite, a nematode parasite, a trematode parasite, a microsporidial parasite, an algal parasite, a plant parasite, an animal parasite, downy mildew, Bremia, Hyaloperonospora, Peronospora, Sclerospora, Peronosclerospora, Sclerophthora, Albugo, Puccinia, Phakopsora, Magnaporthe, Gaeumannomyces, Synchytrium, Ustilago, Tilletia, Erysiphe, Blumeria, Fusarium, Leptosphaeria, Coccidioides, Paracoccidioides, Pneumocystis, Histoplasma, Cryptococcus, Plasmodium, Babesia, Cryptosporidium, Toxoplasma, Trypanosoma, Leishmania, Giardia, Enterocytozoon, and Encephalitozoon, Triphysaria, Striga, Cuscuta. Human Immunodeficiency Virus, influenza virus, Epstein-Barr Virus, varicella-zoster (chicken pox) virus, hepatitis B virus, adenovirus, any pox virus, variola major (smallpox) virus, any hemorrhagic fever virus, Ebola virus, Marburg virus, Lassa fever virus, Crimean-Congo hemorrhagic fever virus any arenavirus, lymphocytic choriomeningitis arenavirus, Junin virus, Machupo virus, guanarito virus, any bunyavirus, rift valley fever bunyavirus, any hantavirus, any flavivirus, dengue virus, any filovirus, any calicivirus, hepatitis A virus, any encephalitis virus, west nile virus, lacrosse virus, California encephalitis virus, Venezuelan equine encephalitis virus, eastern equine encephalitis virus, western equine encephalitis virus, Japanese encephalitis virus, Kyasanur forest virus, yellow fever virus, rabies virus, Chikungunya virus, severe acute respiratory syndrome-associated (SARS) coronavirus, Francisella, Burkholderia, Coxiella, Brucella, Chlamydia, Mycobacterium, any Rickettsia, Rickettsia prowazekii (Typhus fever), Listeria, Cyclospora, and Entamoeba.

7. The fusion construct of claim 2, wherein
said pathogen is an oomycete;
said characteristic lipid is phosphatidylinositol-3-phosphate (PI-3-P) or phosphatidylinositol-4-phosphate (PI-4-P);
said at least one first domain comprises a protein or polypeptide specific or selective for binding to said PI-3-P or said PI-4-P; and
said at least one second domain is toxic or inhibitory to said oomycete.

8. The fusion construct of claim 1, wherein said characteristic lipid is selected from the group consisting of proteolipids, glycolipids, sphingolipids, phospholipids, sulfolipids and sterols.

9. The fusion construct of claim 6, wherein said characteristic lipid is selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), lysophosphatidyl-inositol-3,4,5-triphosphate (LPI-3,4,5-P3), phosphatidyl-inositol (PI), lysophosphatidyl-inositol (LPI); phosphatidyl-serine (PS), phosphatidyl-glycerol (PG), phosphatidyl-ethanolamine (PE), phosphatidyl-choline (PC), lysophosphatidyl-serine (LPS), lysophosphatidyl-glycerol (LPG), lysophosphatidyl-ethanolamine (LPE), lysophosphatidyl-choline (LPC), phosphatidic acid (PA), lysophosphatidic acid (LPA), sphingosine-1-phosphate (S-1-P), ceramide-1-phosphate (C-1-P), a glycosylphosphatidylinositol (GPI)-protein anchor, a galactolipid, a glycoceramide, glucosyl-ceramide, galacto-ceramide, glycosylsphingosylinositol (GSI), glycosyl phosphoryl inositol ceramide (GPIC), sphingomyelin (SM), and ergosterol.

10. The fusion construct of claim 1, wherein said at least one first domain comprises a moiety selected from the group consisting of: a pleckstrin homology (PH) domain; a protein kinase C domain 1 homology (C1) domain; a protein kinase C domain 2 homology (C2) domain; a Fab 1, YotB, Vac 1 and EEA1 homology (FYVE) domain; a Phagocytic oxidase homology (PX) domain; an Epsin N terminal Homology (ENTH) domain; a Bin-Amphiphysin-Rvs (BAR) domain; a Four point one protein; Ezrin, Radixin and Moesin homology (FERM) domain; a post synaptic density 95 protein; Drosophila disc large tumor suppressor A and Zonula occludens 1 homology (PDZ) domain; a tubby protein homology (tubby) domain; a defensin; a cathelicidin; and a lipid transfer protein.

11. The fusion construct of claim 1, wherein said at least one first domain comprises a moiety selected from the group consisting of: human phosphatidylinositol-4-phosphate adaptor protein-1 (FAPP1) PH domain, a human phosphatidylinositol-3-phosphate-binding PH-domain protein-1 (PEPP1)-PH domain, an Arabidopsis-PH-domain-protein-1 (AtPH1) PH domain, a soybean AtPH1-homolog (GmPH1) PH domain, an Arabidopsis Enhanced Disease Resistant-2 (EDR2) PH domain, an Arabidopsis phosphatidylinositol-4-kinase (PI4K) PH domain, a potato EDR2 PH domain, a tobacco PI4K PH domain, a soybean EDR2 PH domain, a soybean PI4K PH domain, Raphanus sativus Anti-Fungal Peptide-2 RsAFP2, Dahlia merckii Anti-Microbial Peptide (DmAMP1), and defensin Bombyx mori cecropin B.

12. The fusion construct of claim 1, wherein said at least one second domain with an activity of interest binds to or covalently modifies a protein of said cell.

13. The fusion construct of claim 1, wherein said at least one second domain with an activity of interest binds to or covalently modifies a nucleic acid of said cell.

14. The fusion construct of claim 1, wherein said at least one second domain with an activity of interest binds to or covalently modifies a lipid of said cell.

15. The fusion construct of claim 1, wherein said at least one second domain with an activity of interest binds to or covalently modifies a carbohydrate of said cell.

16. The fusion construct of claim 1, wherein said at least one second domain with an activity of interest binds to or covalently modifies a small molecule within said cell.

17. A method of delivering a substance of interest to a cell, comprising the step of
contacting said cell with a fusion construct comprising
at least one first domain specific or selective for binding to a characteristic lipid on the surface of said cell; and
at least one second domain comprising said substance of interest.

18. The method of claim 17, wherein said at least one second domain comprising said substance of interest is capable of modifying the metabolism, physiology, development or growth of said cell.

19. The method of claim 17, wherein said cell is a pathological cell displaying a characteristic lipid and said substance of interest is a therapeutic substance that remedies the pathological functions of said cell.

20. A method of killing, damaging or inhibiting a pathogenic cell, a cancer cell or other pathological cell displaying a characteristic lipid, comprising the step of
contacting said pathogenic cell, said cancer cell or said other pathological cell displaying a characteristic lipid with a fusion construct comprising
at least one first domain specific or selective for binding to said characteristic lipid on a surface of said pathogenic cell, said cancer cell or said other pathological cell displaying said characteristic lipid; and
at least one second domain capable of killing, damaging or inhibiting said pathogenic cell, said cancer cell or said other pathological cell displaying said characteristic lipid.

21. The method of claim 20, wherein said pathological cell is a host cell infected by a pathogen.

22. The method of claim 20, wherein said pathogen is of a type selected from the groups consisting of a virus, an archaebacterium, a bacterium, a fungus, an oomycete, an apicomplexan parasite, a trypanosomatid parasite, an amoebozoan parasite, a nematode parasite, a trematode parasite, a microsporidial parasite, an algal parasite, a plant parasite, an animal parasite, downy mildew, Bremia, Hyaloperonospora, Peronospora, Sclerospora, Peronosclerospora, Sclerophthora, Albugo, Puccinia, Phakopsora, Magnaporthe, Gaeumannomyces, Synchytrium, Ustilago, Tilletia, Erysiphe, Blumeria, Fusarium, Leptosphaeria, Coccidioides, Paracoccidioides, Pneumocystis, Histoplasma, Cryptococcus, Plasmodium, Babesia, Cryptosporidium, Toxoplasma, Trypanosoma, Leishmania, Giardia, Enterocytozoon, and Encephalitozoon, Triphysaria, Striga, Cuscuta. Human Immunodeficiency Virus, influenza virus, Epstein-Barr Virus, varicella-zoster (chicken pox) virus, hepatitis B virus, adenovirus, any pox virus, variola major (smallpox) virus, any hemorrhagic fever virus, Ebola virus, Marburg virus, Lassa fever virus, Crimean-Congo hemorrhagic fever virus any arenavirus, lymphocytic choriomeningitis arenavirus, Junin virus, Machupo virus, guanarito virus, any bunyavirus, rift valley fever bunyavirus, any hantavirus, any flavivirus, dengue virus, any filovirus, any calicivirus, hepatitis A virus, any encephalitis virus, west nile virus, lacrosse virus, California encephalitis virus, Venezuelan equine encephalitis virus, eastern equine encephalitis virus, western equine encephalitis virus, Japanese encephalitis virus, Kyasanur forest virus, yellow fever virus, rabies virus, Chikungunya virus, severe acute respiratory syndrome-associated (SARS) coronavirus, Francisella, Burkholderia, Coxiella, Brucella, Chlamydia, Mycobacterium, any Rickettsia, Rickettsia prowazekii (Typhus fever), Listeria, Cyclospora, and Entamoeba.

23. A method of delivering a substance of interest to a target cell, comprising the step of
contacting a host cell containing said target cell with a fusion construct comprising
at least one domain that binds to a surface of said host cell;
at least one first domain specific or selective for binding to a characteristic lipid on the surface of said target cell; and
at least one second domain comprising said substance of interest.

24. The method of claim 23, wherein said at least one domain specifically or selectively binds to a characteristic lipid on said surface of said host cell.

25. A method of killing, damaging or inhibiting a pathogenic cell located within a host cell, comprising the step of
contacting a host cell containing said pathogenic cell with a fusion construct comprising
at least one domain that binds to a surface of said host cell;
at least one first domain specific or selective for binding to a characteristic lipid on the surface of said pathogenic cell; and
at least one second domain capable of killing, damaging or inhibiting said pathogenic cell.

26. The method of claim 25, wherein said at least one domain specifically or selectively binds to a characteristic lipid on said surface of said host cell.

27. A plant, animal or microbial cell that is genetically modified to contain and express nucleic acid sequences encoding
a protein construct comprising
at least one first domain specific or selective for binding to a characteristic lipid on the surface of a target cell; and
at least one second domain with an activity of interest.

28. The plant, animal or microbial cell of claim 27, wherein said target cell is a microbial cell.

29. The plant, animal or microbial cell of claim 27, wherein said target cell is a symbiotic cell and said plant, animal or microbe is a host of said symbiotic cell.

30. The plant, animal or microbial cell of claim 29, wherein said symbiotic cell is mutualistic with, commensal on, or pathogenic on said host plant, animal or microbe.

31. The plant, animal or microbial cell of claim 29, wherein said at least one second domain with an activity of interest alters the metabolism, physiology, development or growth of said symbiotic cell.

32. The plant, animal or microbial cell of claim 29, wherein said symbiotic cell is pathogenic and said at least one second domain kills, damages or inhibits said symbiotic cell.

33. A method of killing or inhibiting a pathogen, a cancer cell, or a pathological cell displaying a characteristic lipid, comprising the step of
contacting said pathogen with a single domain agent which binds to a characteristic lipid on a surface of said pathogen, said cancer cell, or said pathological cell displaying a characteristic lipid and interferes with said characteristic lipid, and wherein interference kills or inhibits said pathogen, said cancer cell or said pathological cell displaying a characteristic lipid.

34. The method of claim 33, wherein said pathogen is an oomycete and said characteristic lipid is phosphatidylinositol-4-phosphate (PI-4-P).

35. The method of claim 33, wherein said cancer cell or other pathological cell is a host cell infected by a pathogen.

36. The method of claim 34, wherein said agent is a phosphotidylinositol-specific phospholipase C.

37. A plant, animal or microbial cell that is genetically modified to contain and express nucleic acid sequences encoding a protein that
specifically or selectively binds to a characteristic lipid on the surface of a pathogen and
exhibits an activity which interferes with a function of said characteristic lipid.
